(19) United States
(12) Patent Application Publication
(10) Pub. No.: US 2013/0018479 A1
(43) Pub. Date: Jan. 17, 2013

(54) RESILIENT INTERPOSITIONAL HIP ARTHROPLASTY DEVICE

(71) Applicant: R. Thomas Grotz, Novato, CA (US)

(72) Inventor: R. Thomas Grotz, Novato, CA (US)

(21) Appl. No.: 13/574,517

(22) PCT Filed: Jan. 19, 2011
(86) PCT No.: PCT/US2011/021673

§ 371 Date: Oct. 8, 2012

Related U.S. Application Data
(60) Provisional application No. 61/297,697, filed on Jan. 22, 2010.

(51) Int. Cl. A61F 2/36

(52) U.S. Cl. 623/22.14

(57) ABSTRACT

This disclosure is directed to a resilient interpositional arthroplasty implant for application into joints to pad cartilage defects, cushion joints, and replace or restore the articular surface, which may preserve joint integrity, reduce pain and improve function. The implant may endure variable joint compressive and shear forces and cyclic loads. The implant may repair, reconstruct, and regenerate joint anatomy, and thereby improve upon joint replacement alternatives. Rather than using periosteal harvesting for cell containment in joint resurfacing, the walls of this invention may capture, distribute and hold living cells until aggregation and hyaline cartilage regrowth occurs. The implant may be deployed into debrided joint spaces, molding and conforming to surrounding structures with sufficient stability to avoid extrusion or dislocation. Appendages of the implant may repair or reconstruct tendons or ligaments, and an interior of the implant that is inflatable may accommodate motions which mimic or approximate normal joint motion.

### CROSS REFERENCE

This application claims the benefit of U.S. Provisional Application No. 61/297,697, filed Jan. 22, 2010 which is incorporated herein by reference in its entirety.

### BACKGROUND OF THE INVENTION

This invention relates to arthroplasty, and more particularly, to an implant for use in arthroplasty when hyaline articular cartilage is damaged, it breaks down and joint space is lost. Inflammatory enzymes such as from the Cox-1, Cox-2 and/or 5-Lox systems, are released and loose bodies form adding to the degradation of joint function. Such joint damage is conventionally treated by physical therapy, analgesics, pain medication and injections. When these treatments fail, the traditionally accepted treatment option is arthroplasty implantation or replacing the joint with an artificial joint construct. Current arthroplasty techniques typically use “plastic and metal” implants that are rigid and which ultimately fail due to loosening or infection. Conventional materials for the artificial joint components include chrome-cobalt-molybdenum alloy (metal) and high molecular weight polyethylene (plastic). Each is often fixed by a cement-like mixture of methyl methacrylate to the ends of the bones that define the joint that is the subject of the arthroplasty, or coated with a surface that enables bone ingrowth. Current hip joint replacements typically last about 10-15 years.

Conditions requiring arthroplasty include traumatic arthritis, osteoarthritis, rheumatoid arthritis, osteonecrosis, and failed surgical procedures.

### SUMMARY OF THE INVENTION

The present invention is directed to an orthopedic implant configured for deployment between opposing members of a joint structure that addresses many of the shortcomings of prior artificial joints. The arthroplasty implants embodying features of the invention are configured to preserve joint motions while removing the pain and dysfunction following the development of arthritis or joint injury. The arthroplasty implant in accordance with the present invention achieves improved physiologic motion and shock absorption during gait and acts as a resilient spacer between moving bones during limb movement. The combined characteristics of the implant include anatomic design symmetry, balanced rigidity with variable attachment connections to at least one of adjacent normal structures, and durability which addresses and meets the needs for repair or reconstruction thus far missed in the prior art. The implant should be secured to at least one of the bones of the joint structure.

Hip patients may requirement treatment of the femoral head and/or acetabular cup cartilages, and/or labral fibrocartilages. Interpositional arthroplasties (such as the implants and methods provided herein) intend to renew joint space, and provide painless gliding motion with clinical need considerations.

Provided herein is a resilient implant for implantation within a ball and socket hip to act as a cushion allowing for renewed hip joint motion.

Provided herein is a hip implant configured for deployment between a femur head and a acetabulum of a hip joint, the implant comprising a balloon comprising a first portion that is configured to engage the femur head of the hip joint, a second portion that is configured to engage the acetabulum of the hip joint, a side portion connecting the first portion and the second portion, in which the side portion facilitates relative motion between the first portion and the second portion, and an interior that is optionally inflatable with a first inflation medium; and a first appendage configured to couple the balloon to the femur head of the joint.

In some embodiments, at least two of first portion, the second portion, and the side portion are contiguous. In some embodiments, the first portion comprises a first wall, the second portion comprises a second wall, and the side portion comprises a side wall.

In some embodiments, the implant comprises an inflation port in communication with the interior of the balloon for inflation of the interior of the balloon with the first inflation medium. In some embodiments, the balloon may be punctured to inflate the interior of the balloon with the first inflation medium. In some embodiments, the balloon is self-sealing. In some embodiments, the balloon is self-sealing upon inflation of the interior of the balloon with the first inflation medium. In some embodiments, the implant comprises a seal capable of closing the interior of the balloon.

In some embodiments, the interior comprises a plurality of inflatable chambers. In some embodiments, the interior comprises a plurality of individually inflatable chambers. In some embodiments, a first chamber of the plurality of individually inflatable chambers is adapted to be inflated with the first inflation medium, and a second chamber of the plurality of individually inflatable chambers is adapted to be inflated with a second inflation medium. In some embodiments, the first inflation medium imparts rigidity in the implant. In some embodiments, the first inflation medium imparts cushion in the implant.

In some embodiments, the interior comprises a honeycomb structure. In some embodiments, the interior comprises a mesh structure. In some embodiments, the interior comprises a sponge structure.

In some embodiments, the implant comprises a second appendage coupling the balloon to the femur head of the joint. In some embodiments, the implant comprises a second appendage coupling the balloon to the acetabulum of the joint. In some embodiments, the implant comprises a second appendage configured to couple at least one of the first portion, the second portion, and the side portion to at least one of the femur head and the acetabulum of the hip joint. In some embodiments, the first appendage and the second appendage are configured to provide ligamentary-like support to the femur head and the acetabulum of the hip joint. In some embodiments, the first appendage and the second appendage are configured to provide ligamentary-like support to the hip joint.

In some embodiments, the implant is configured to fit within a cannula having a distal end inner diameter of at most 10 millimeters. In some embodiments, the implant is configured to fit within a cannula having a distal end inner diameter of at most 9 millimeters. In some embodiments, the implant is configured to fit within a cannula having a distal end inner diameter of at most 5 millimeters. In some embodiments, the implant is configured to fold in order to fit within a cannula having a distal end inner diameter of at most 10 millimeters. In some embodiments, the implant is configured to fold in order to fit within a cannula having a distal end inner diameter of at most 9 millimeters. In some embodiments, the implant is configured to fold in order to fit within a cannula having a distal end inner diameter of at most 5 millimeters. In some embodiments, the implant is configured to be delivered to a joint through a cannula having a distal end inner diameter of at most 10 millimeters. In some embodiments, the implant is configured to be delivered to a joint through a cannula having a distal end inner diameter of at most 9 millimeters. In some embodiments, the implant is configured to be delivered to a joint through a cannula having a distal end inner diameter of at most 5 millimeters.

In some embodiments, the implant replaces periosteum.

In some embodiments, the implant is configured to at least one of: pad cartilage, cushion the joint, deliver a pharmacologic substance, remove noxious enzymes, debride upon implantation, debride the joint following implantation, deliver a therapeutic substance, deliver a biologic substance, and deliver living stem cells. In some embodiments, the implant is configured to deliver a chemotherapeutic agent to a bone or other surrounding tissues. In some embodiments, the implant is configured to deliver an anti-infectious medication to a bone or other surrounding tissues. In some embodiments, the implant is configured to deliver at least one of an antibiotic, antifungals, and analgesics agent.

In some embodiments, the implant is configured to be selectively inflated to realign limbs.

Provided herein is a method comprising: implanting a hip implant as described herein into a subject, wherein the implant reverses arthritis in the subject.

Provided herein is a method comprising: implanting a hip implant as described herein into a hip joint of a subject and treating a component of the hip joint of the subject with at least one of an allograph tissue, an autograph tissue, and an xenograph tissue. In some embodiments, the implanting step is at least one of: prior to the treating step, simultaneous with the treating step, and following the treating step.

Provided herein is a method comprising: implanting a hip implant as described herein into a subject, wherein the implant at least one of: restores joint function and controls arthopathies. In some embodiments, the implanting spares existing anatomy.

Provided herein is a method comprising: debriding a femur head of a hip joint of a subject, and implanting a hip implant as described herein into the hip joint of the subject, whereby the implant is configured to anneal to the cartilage of the subject. In some embodiments, the debriding is achieved by steam application.

Provided herein is a method comprising implanting a hip implant as described herein into a joint previously treated with a total joint replacement. In some embodiments, the method comprises removing the total joint replacement prior to implanting the hip implant. In some embodiments, the method comprises clearing infectious matter from the joint and/or surrounding tissues. In some embodiments, the method comprises implanting a second implant of any implant described herein following removing the implant previously implanted in the joint. In some embodiments, the method comprises replacing the joint of the subject following removing the implant previously implanted in the joint. In some embodiments, the method comprises debriding the bone of the joint, and implanting an implant of any implant described herein. In some embodiments, the method comprises repeating the debriding and implanting steps.

These and other advantages of the invention will become more apparent from the following detailed description and the attached exemplary drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view, partially in section, of an implant embodying features of the invention with an enlarged upper portion prior to implantation.

FIG. 2 is an elevational view of the implant shown in FIG. 1 mounted on the head of a patient's femur.

FIG. 3 is a cross-sectional view of the implant shown in FIGS. 1 and 2 deployed between the head of a patient's femur and acetabulum after release of traction to allow for the bones to settle into their natural albeit pathologic angles of repose.

FIG. 4 is an elevational view of a resilient arthroplasty implant with a smaller upper portion than that shown in FIGS. 1-3 that has been deployed between the head of patient's femur and the acetabulum of the pelvic bone.

FIG. 5 is an elevational anterior view of a left proximal femur with an implant placed over the femoral head portion of the hip joint as shown in FIG. 4, in partial cross section, to illustrate details thereof.

FIG. 6 is a lateral elevational view of a femur with the implant shown in FIG. 4, as viewed from the “side of the body” or lateral hip aspect.

FIG. 7 is a superior view of a femur with the implant shown in FIG. 4.

FIG. 8 is an inferior view of the hip joint invention iteration or implant in FIG. 7.

FIG. 9 is a superior or cephalad view of a patient's hip with a resilient implant having features of the invention, viewed from the head of the patient or from a cephalad to caudad direction.

FIG. 10A depicts an embodiment of the implant having an appendage that is in the form of a skirt and a balloon that is mounted on a femur head and implanted in the space between the femur head and the acetabulum of the pelvic bone.

FIG. 10B depicts an embodiment of the implant having appendages (tab type) and a balloon that is mounted on a femur head and implanted in the space between the femur head and the acetabulum of the pelvic bone.

FIG. 11A depicts an embodiment of the implant having appendages (tab type) and a balloon that is mounted on a femur head wherein the balloon is minimally inflated (or not inflated). FIG. 11B depicts an embodiment of the implant having appendages (tab type) and a balloon that is mounted on a femur head wherein the balloon is minimally inflated (or not inflated) and showing a tube that may be used to inflate the balloon of the implant or to extract inflammatory enzymes. FIG. 11C depicts an embodiment of the implant having appendages (tab type) and a balloon that is mounted on a femur head wherein the balloon is inflated and showing an inflation tube.

FIG. 12 depicts an embodiment of the implant having appendages (tab type) and an inflated balloon that is mounted on a femur head and implanted in the space between the femur head and the acetabulum of the pelvic bone.

### DETAILED DESCRIPTION OF THE INVENTION

The present invention is directed to arthroplasty implants and methods for a hip.

Some embodiments of the implant comprise a balloon, or bladder, as an interpositional arthroplasty of the human and animal joint that recreates cartilage once damaged. The implant may conform once inflated to internal joint components, for example into the interstices of joint opposing surfaces.

The hip is more simple than some other joints (such as the knee) since the hip has only one one cartilage/space/cartilage-bone interface (which equal a joint.)

Hip pain is one of the most common arthritities affecting humans, and it manifests in the groin with pain, grinding, immobility and throbbing discomfort. Each person has his own pain tolerance level, and ways of dealing with the situation. Some people can tolerate pain simply by ‘letting the pain pass by’ yet others are mentally and physically incapacitated by pain. Embodiments of the device described herein fill the gap between the femoral head (ball) and the acetabular (cup) relieving hip pain and discomfort by restoring the cushion in the joint and restoring function.

Diagnosis of the hip condition involves a history from the patient that reports usually progressive but occasionally abrupt and then consistent onset of groin pain (half way between the genitals and lateral most aspect of the hip) wherein aching, grinding (=crepitus), giving way (if there is a labral tear), throbbing awakening one from sleep, or rarely acute onset (as from infections like syphilis or metabolic problems like gout or avascular necrosis) lead to breakdown of the joint surface interval. Normally the femoral head and acetabular cartilage are about 2-3 mm thick, involving histologically unique hyaline layers of white shiny smooth gliding lubricious low modulus opposing surfaces, however, in pathology the surfaces become disrupted. The patient complains of pain in the groin. If it is lateral, where one's hip contacts the bed at nighttime lying on one's side with pain induced by palpating the lateral hip, the diagnosis is more likely bursitis and not related to the joint. In the case where pain in ‘the hip’ comes from the back coursing obliquely around the lateral hip thru the groin to the mid thigh, which may be L2 nerve root (spinal) impingement.

X-rays may show either a loose body or radio-opacity about a cm in diameter, like a small snow ball of collected boney cartilaginous debris that settles in the joint capsular area. Normally the joint space on X-ray is about 8 mm, but as narrowing diffusely develops from rheumatoid or osteoarthritis the joint space narrows, especially on standing films, when disparity can be seen in comparison to the contralateral (if normal) hip. MRI scans can show the Ficat I-IV or Glimshire I-VI stages of avascular necrosis (death of bone) that is usually idiopathic but sometimes epidemiologically related to alcohol or steroid use, or with scuba divers Caison's disease can develop. Regardless, the signs and symptoms are usually at the anterior center of the hip.

The Physical Exam normally allows for hip flexion of 120 degrees, extension of 20, abduction, external and internal rotation of 45 degrees, and adduction of 20. With hip disease or injury the Range of Motion is frequently decreased, and tenderness is anterior. A limp with gait may be visible.

Treatments range from time for healing, activity modification, physical therapy, medications (topical, oral non-steroidal anti-inflammatories aimed at the Cox-1, Cox-2, and/or 5-Lox enzyme systems), analgesics, muscle relaxants, injections (steroids . . . discouraged as they can degrade cartilage or start an infection, or viscolubricants as Synvisc or Hyalgan, followed by arthroscopy (done only by few and experienced surgeons gaining hip joint access under general anesthesia and 60 pounds special traction), leading to total joint replacement arthroplasty. While hip joint replacements as a last resort to deter severe groin pain and loss of function in activities of daily living can reduce or ablate pain for walking for 10-20 years, invariably the implant ultimately fails as does every physical thing. Hip arthroplasties fail due to either loosening or infection. Revision surgery is fraught with more bone resection, and when infected with IV antibiotics for use over a 6 week to 6 month period when cement implanted with antibiotics is left in the hip joint (preventing movement.) Even patients with ideal results in THRs (total hip replacements) are still at risk of dislocation with hip flexion and internal rotation (as when tying one's shoes). In that case severe pain accrues, the patient cannot walk and must be transported to the ER/OR usually for a general anesthetic and reduction/realignment of implanted parts. Once a dislocation occurs, since the joint capsule stretches a repeat episode is more likely.

Total hip replacements typically require open surgical procedures with incisions of 4-12 inches, and having a surgical duration of about 2-4 hours. Additionally, there is little bone and cartilage preservation, extensive soft tissue dissection and dislocation of the hip during the surgery thus the normal anatomy of the joint is not retained. Such dislocation can result in disruption of the central and other ligaments and the stabilizing capsule as well as the blood supply to the femoral head that enters the neck of the femur with the anterior and posterior circumflex arteries. Once dislocation occurs (whether traumatically or for ‘treatment’,) the possibility of femoral head bone death (i.e. avascular necrosis) is increased. The total hip replacement option for patients is poorly adapted to revision surgery, and often results in limitations to joint function and the natural stride of the patient is not typically retained. In total replacement surgeries, time back to work is about 6 weeks, where total time for recovery is more on the order of one year. The implants are often metal resulting in metal detection issues. Post-op hospitalization can last 3-6 days, and the treatment cost can be about $250 k over the patient's lifetime.

As an alternative, capping or resurfacing treatments are currently available and including treatments having metal placed over a ground-down cartilage surface wherein the metal articulates with the remnant cup cartilage per se. In some of these procedures, the metal femoral head cover opposing a metal cup insert. Regardless, much like the total joint replacement surgery, the hip still needs to be dislocated, with similar consequences as noted in regard to the total hip replacement option. In such procedures, the surgery is a open procedure lasting about 2-4 hours and requiring incisions of 4 to 12 inches typically. There is extensive soft tissue dissection. There is some limitation to joint function, however the natural stride is typically not retained. The time back to work may be about 6 weeks, with about 6-10 months until total recovery is achieved. The post-operation hospitalization is typically 2-4 days, and the treatment cost for this procedure over the patient's lifetime can be upwards of about $100 k.

Unlike other hip joint treatments in which the hip needs to be dislocated and potentially, embodiments of the implant provided herein do not require dislocation for proper placement of the implant. Some embodiments of the implant described herein can be inserted using incisions that are at most one half inch each. In some embodiments, the surgical duration is about 1 hour, and only non-functional tissue (bone and/or cartilage) is removed—thus preserving functional tissues (or most). In some embodiments, there is only minimal soft tissue dissection required, and the implant is highly adaptable to revision surgery. In some embodiments, there results in minimal limitation to joint function, and the natural stride of the patient can be preserved. In some embodiments, the time back to work can be in a few days, and the recovery time can be less than a few months. Likewise, some embodiments only require an outpatient procedure and thus costs of embodiments of the device over the patient's lifetime can be less than other options currently available.

Some embodiments will be used in conjunction with arthroscopic debridement. Arthroscopic debridement of the hip is a specialized treatment, required usually imported traction systems that put a crutch like curved padded handle in the mid pelvic region (as beneath the scrotum) and then the leg is attached to a traction or pulling device that stretches the hip joint from its normal 5-6 mm to a radiographic or image intensifier illustrated 10-12 mm on AP view on general anesthesia) so that arthroscopic cannulae can enter the hip joint, visualize it fiberoptically on the OR TV screen, and debride the joint. This set up and debridement can in some embodiments be a predecessor to implant placement, which may require one quarter inch (5-6 mmd) and one half inch (10-12 mm) periarticular wound (incision) using a 3-M pump (so as to avoid the need for a third hip arthroscopic incision). In the hip arthroscopic four processers are engaged 1) loose bodies are removed, 2) synovitis is ablated mechanically and electronically 3) the remnant hyaline cartilage femoral head and acetabular interfaces are viewed, assessed, potentially prepared with stable defect edges and 4) labrum fibrocartilage (meniscus-like) tears are trimmed

Once the arthroscopy is done, if the surgeons stops there, a pain reduction and functional improvement may be expected for 3-6 months, especially in a viscolubricant is added. However, the practice of providing hip arthroscopies for arthritic hips as a routine treatment has been progressively discouraged as in the VA Study, as the relief from symptoms is quite brief, and not a curative effort.

The jump in required therapy to a total joint replacement or resurfacing arthroplasty is major. In current such procedures an incision lateral and/or anterior 10-20 inches in length down to the hip joint is needed. The femoral head is sacrificed and ligaments are removed and sometimes reattached (though usually resulting in a typical limp or Trendelenburg gait). The femoral head cartilage is reemed. Screws are placed into the cup or it is cemented into the groin with methymethacrylate. The hip stem is either jammed into the femur in hopes bone ingrowth will eventually occur, or cemented (which usually eventually loosens). The metal stem is then attached via a (Morse or C) taper to a metal ball than is hammered on, so it usually ‘sticks onto the stem.’ Once the metal cup is in the pelvis a white plastic high molecular weight polyethylene which is hard, almost with a durometry resembling metal, is inserted into the metal acetabular cup. The stem and ball are then relocated into the pelvic new artificial cup, and a ligament and capsular repair begin. The patient remains at risk of dislocation or infection there after.

In summary existing art and science stop at arthroscopy and the treatment gap extends to complete ablation of the joint. The missing tools are interpositional arthroplasties, which embodiments of the implants described herein provide. Indications for hip implants are failed treatment conservatively in patients toward or through arthroscopic debridement, when pain and dysfunction require further surgical care. Patients who are younger, intend to enjoy physiologic normal activities, do not wish to succumb to joint ablation, to procedures sacrificing their normal cartilage, bone, ligaments and capsules, to major blood loss, to permanent risks of infection, of dislocation, and to ‘bridge burning’ that precludes later reconstruction or ‘arthritis reverse’ should consider joint balloons as an opportunity to temporarily or permanent restore the normal cartilage interfaces. Contraindications to implant uses in some embodiments include active infection of the ipsilateral joint, allergy to the polymer of implant make up, and advanced joint deformity with instability that will otherwise require osteotomy or complicated reconstructive efforts that lead to prosthetic implants such as bipolar hemiarthroplasties that do not dislocate under normal circumstances.

In some embodiments, the implant may be selectively inflatable depending on the particular needs of the patient. In some embodiments, the filler of the interior of the implant may be rigid, semi-rigid, fluid, air, or combinations thereof, as described herein. In some embodiments, the implant may be used in conjunction with fibrocartilage repair or replacement. In some embodiments, the implant may be used without fibrocartilage repair or replacement. In some embodiments, the implant may be used in conjunction with boney osteotomy. In some embodiments, the implant may be used without boney osteotomy.

The preparation, anesthesia, joint distraction, precautions regarding infection and noxious pressures upon soft tissue structures involved with the traction device, and general arthroscopic hip debridement are the same for implant placement (that is, regardless of whether the ‘balloon’ is inserted). As the joint is prepared special attention is given to Grade III and IV cartilage defects that may benefit from chondrocyte insertion so that ragged edges are made stable to the extent that the implant intends to deliver cartilage chondrocytes or stem cells with or without adjunct pharmacologies growth inducing/anti-inflammatory/anti-infectious/viscolubricant/inflative (cushioning/padding) agents so as to restore the joint surfaces and interfaces toward normal.

Certain embodiments of the implant can be inserted through cannulas having obturators (for non-limiting example, the Smith and Nephew, Inc. Acufex 10 mm×756 mm Clear-Trac threaded Cannula with 4 mm Cannulated Obturator). However, some implant insertions may require larger incisions as through wounds up to 10 or more cm for application of the balloon or polymer cover to the femoral head. To the extent arthroscopic facilitation can be used, the implant will be inserted deflated, will be draped over the femoral head with or without a clearance (or slot) to accommodate the ligamentum flavum, will be tacked down via sutures and suture anchors, staples, screws, Stabilizers, and/or other couplers described herein, considering the natural anatomy and implant compliance, benefitting from intended design concurrent non-compliant hemisphere shaped features, to produce a coverage shaped like a hemisphere (resembling the rind of half an orange) to drape over the upper weight bearing surface of the femoral head. Some embodiments apply the implant to the larger adjacent radius of the femoral cup or acetabulum and or to opposing surface.

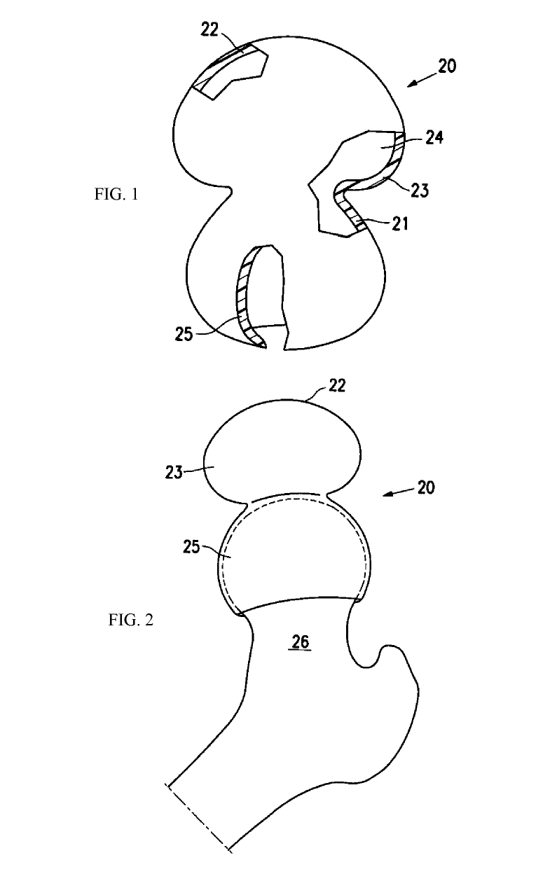

Some embodiments cover the head of the femur directly, with a radius or surface that is attached to the remnant ball, that fills in the defects of that hyaline surface with padding and/or restorative cells, fixed to the ball (or optionally to the cup), and the other implant radius will be free moving. Some embodiments comprise a large redundant membrane that in and of itself rolls with normal hip joint motion. In some embodiments, the redundant membrane may serve not only to enhance natural motion between variable layers but also to restore stability normally provided by the fibrocartilage rim around the acetabular hyaline cartilage periphery. Such a redundant membrane is shown, for example, in FIGS. 1-3, at least. The implant in some embodiments will be succinct and without redundancy to have the smaller radius fixed to the ball, and the larger radius mobile and gliding against the socket. There will be a trend to try to preserve all normal tissues in embodiments of the methods and implants described herein.

Hip signs and symptoms after implantation are expected to report reduced pain and improved function. As the ligaments are preserved and incisions are tiny, there is no expectation that dislocation may occur. Blood loss should be negligible. Time for surgery should be short. Complications can be dealt with in outpatient surgery usually. In the case of infection or implant disruption due to secondary trauma, extraction can be accomplished still leaving existing conservative treatment and/or joint arthroplasty options. The deflated implant of certain embodiments is shaped like the upper half of the femoral head and may be inserted through the smallest possible incisions, secured to avoid dislocation, inflated with minimal amounts of air/gel/liquid to accommodate glide and proper fixation, and the patient will enjoy a restored joint surface.

Ideally the implant will be left in place indefinitely without failing, and without immediate or late noxious effects such as the silicon synovitis of the past. However, options include application of the balloon or implant as a temporarily balloon to deliver pharmacologic substances including medications and stem cells for as little as 23 hours, so that the cells can attach in the same time period realized for existing FDA cleared Genzyme Carticel chondrocytes. In some embodiments of implant use, the massively disruptive periosteal dissections and harvesting through as much as a four foot long incision will no long be needed, since the implant polymer will be the ‘man hole cover’ or container for the first 23 hours while cells attach whereupon the implant cover may be removed. Better will be the use of the polymers for padding, cushioning and physiologic restorative treatment for 27 days until wounds heal.

In some embodiments, the implant is biodegradable (in part or in whole).

In some embodiments, the implant comprising an inflatable balloon will be inserted. The implant in some embodiments is configured to cushion the joint enduring the 6-8 times body weight compressive and shear forces, the millions of cyclic loads, and the other requirements that enable treated patients to acquire the best feasible quality of life.

Some embodiments of the implant will serve to add padding to the debrided hip joint to remove pain and improve function via stable inflatable interpositional arthroplasty placement, thus cushion the articulating structures, and returning new cartilage growth for restoration.

Provided herein is a resilient implant for implantation into a hip joint to act as a cushion allowing for renewed joint motion. The implant may endure variable joint forces and cyclic loads while reducing pain and improving function after injury or disease to repair, reconstruct, and regenerate joint integrity. The implant may be deployed in a prepared debrided joint space, secured to at least one of the joint bones and expanded in the space, molding to surrounding structures with sufficient stability to avoid extrusion or dislocation. The implant may have has opposing walls that move in varied directions, and an inner space filled with suitable filler to accommodate motions which mimic or approximate normal joint motion. The implant may pad the damaged joint surfaces, restores cushioning immediately and may be employed to restore cartilage to normal by delivering regenerative cells.

Provided herein is a resilient interpositional arthroplasty implant for application into a hip joint to pad cartilage defects, cushion joints, and replace or restore the articular surface, preserving joint integrity, reducing pain and improving function. The implant may endure variable joint compressive and shear forces, and millions of cyclic loads, after injury or disease requires intervention. The implant may repair, reconstruct, and regenerate joint anatomy in a minimally morbid fashion, with physiologic solutions that improve upon the rigid existing joint replacement alternatives of plastic and metal. In cases where cells have been used for joint resurfacing requiring massive periosteal harvesting for containment, the polymer walls of some embodiments of the implant can capture, distribute and hold living cells until aggregation and hyaline cartilage regrowth occurs. The implant may be deployed into a prepared debrided joint space, molding and conforming to surrounding structures with sufficient stability to avoid extrusion or dislocation. Appendages of the implant may serve to repair or reconstruct tendons or ligaments. The implant may have opposing walls that move in varied directions, and an inner space, singular or divided, filled with suitable gas, liquid, and/or complex polymer layers as force-absorbing mobile constituents, such than robust valid and reliable joint motion is enabled.

Provided herein is a hip implant configured for deployment between a femur head and acetabulum of a hip joint, the implant comprising a balloon comprising a first portion that is configured to engage the femur head of the joint, a second portion that is configured to engage the acetabulum of the joint, a side portion connecting the first portion and the second portion, in which the side portion facilitates relative motion between the first portion and the second portion, and an interior that is optionally inflatable with a first inflation medium; and a first appendage configured to couple the balloon to the femur head of the joint. The terms “balloon” and “bladder” may be used interchangeably throughout this disclosure to describe an implant having the features described herein.

In some embodiments, at least two of first portion, the second portion, and the side portion are contiguous. In some embodiments, the first portion comprises a first wall, the second portion comprises a second wall, and the side portion comprises a side wall. As used herein, each of the terms the “first portion”, the “second portion”, and the “side portion” is used to describe a part of the balloon, and may not be separate portions in some embodiments. Rather, in some embodiments, each is named in order to indicate the general geometry and location of each portion relative to the other of the portions and/or relative to bones and/or ligaments and/or tendons of the joint. Likewise, as used herein, each of the terms the “first wall”, the “second wall”, and the “side wall” is used to describe a part of the balloon, and may not be separate parts of the balloon in some embodiments. Rather, in some embodiments, each of the walls is named in order to indicate the general geometry and location of each portion relative to the other of the portions and/or relative to bones and/or ligaments and/or tendons of the joint. In some embodiments, at least two of first wall, the second wall, and the side wall are contiguous. Nevertheless, each of the walls may, in some embodiments, be separate parts of the implant that are joined to form the implant. Likewise, each of the portions may, indeed, in some embodiments, be separate parts of the implant that are joined to form the implant.

In some embodiments, the first portion is a term used interchangeably with the first wall. In some embodiments, the second portion is a term used interchangeably with the second wall. In some embodiments, the side portion is a term used interchangeably with the side wall. In some embodiments, a wall (whether a first wall, a second wall, and/or a side wall) of the implant may comprise a plurality of layers. The wall may comprise multiple materials to impart physical and/or therapeutic characteristics to the wall.

Some embodiments of the implant may comprise a first wall, a second wall, and a side wall which define the implant interior (or interior) which contains filling material. In some embodiments, the filling material is an inflation medium. The first wall is secured to the end of the femur head by a skirt that extends from the first wall and the second wall engages the end surface of the acetabulum and may also be secured thereto. In some embodiments, the skirt is called an appendage. The side wall extending between the first and second walls and defines at least in part the implant interior which is filled with filling material (or an inflation medium). The inner surfaces of wall and skirt preferably conform to the particular surface of the femur head. In some embodiments, the inner surfaces of wall and skirt preferably conform to the particular surface of the patient's femur head. The outer surface of the second wall is preferably configured to conform to the end surface of the acetabulum. In some embodiments, the outer surface of the second wall is preferably configured to conform to a surface of the acetabulum.

In some embodiments the attachment elements (also and/or alternatively called coupling elements and/or tabs and/or attachment elements) of the implant comprises holes through which screws or other couplers may be placed to attach the implant to an attachment site (or connection site or coupling site) in the bone of the femur (and/or the acetabulum). In some embodiments, the holes are created arthroscopically. In some embodiments the holes are pre-fabricated in the implant. In some embodiments, the holes may be made prior to implantation based on the patient's particular anatomy. In some embodiments, the holes are reinforced by a reinforcing material of the implant. The reinforcing material may be a polymer of sufficient durometer and/or tear resistance to reinforce the screw hole. The reinforcing material may be comprise metal. In some embodiments, there is no pre-formed hole, but rather screws (or another coupler) secure the attachment tabs (which may be a non-balloon portion of the implant) to the joint component (bone, etc) by creating their own hole when implanted. In some embodiments, the implant may comprise tabs that are adapted to receive staples or other couplers described elsewhere herein.

The implants described herein may comprise attachment elements (or tabs) which may then by attached or coupled to tissue of a component of the joint (whether to a bone or a ligament or a tendon or other joint component) by a coupling device. Coupling devices (or couplers) may comprise at least one of screws, washers, sutures, suture anchors (metal and/or biodegradable), rivots, staples (with and/or without teeth), stabilizers, glues, hooks of cylindrical wire or flattened sheet metal into bone holes or slots respectively. The coupling devices may be resorbable or not. Also, the coupling devices may comprise at least one of strings (i.e. drawstrings), reigns, lassos, and lanyards. The strings, reigns, lassos, and/or lanyards may join with themselves and/or other coupling devices. The couplers provided herein may include a drawstring configured to draw the periphery of the implant around the femoral neck.

In some embodiments, a screw through tab having reinforced center holes may be part of the implant. For example, the implant may comprise polymer covered metal washer holes. The screw may go through the holes. Another embodiment may comprise a staple having spikes. Combinations of spikes and screws may be used in some embodiments, or combinations of other couplers. The implant may be configured to allow a surgeon the option of several types and sizes of couplers, as each patient differs with regard to size and depth of lesion, bone stock, regrowth capability, and compliance with advised recovery, and each surgeon has his own strengths and comforts when working with such implants.

In some embodiments, the implant is configured such that the tabs and/or couplers of the implant couple to the bone where there is no natural cartilage. In some embodiments, the implant may be adapted by the surgeon at the time of surgery such that the tabs are positioned where there is no natural cartilage.

The edge of the implant may have a depending skirt to secure or anchor the implant to the end of bone, but may have one or more depending tabs (or appendages) that may be employed for similar functions as will be discussed in other embodiments. The skirt (and/or tabs, and/or appendages) may tightly fit about the end of the femur head as shown, or the skirt can be secured by adhesive (e.g. methyl methacrylate, bone ingrowth) to the supporting bone structure or be mechanically connected by staples, screws and the like. Moreover, the lower portion of the skirt may be secured by a purse string suture or a suitable strand (elastic or tied) that is tightly bound about the outside of the skirt.

In some embodiments, the implant comprises a methymethacrylate what is placed into a balloon chamber that fits into a bone hole. Such an embodiment would generally fix the implant to the bone once the methymethcrylate cures to a solid.

In some embodiments, the implant can be anchored with generic available sutures and suture anchors fixing and positioning material to bone with proper tensioning.

In addition to the general ingrowth that may occur based on the implant features described herein, the implant undersurface (adjacent the femur head) may comprise an ingrowth matrix. In some embodiments, at least a portion of the implant adjacent to the femur comprises bone ingrowth materials. Such an implant can be attached by a series of tabs with or without holes, using screws, rivots, stabilizers, staples, tacks, or Sutures and suture anchors, for non-limiting example. The polymer of the implant substitutes for periosteum when the implant comprises living chondrocytes (e.g. Carticel) as the ingrowth matrix on a surface of the implant. The polymer of the implant substitutes for periosteum when the implant comprises living chondrocytes (e.g. Carticel) as the ingrowth matrix within an implant embodiment configured to reveal and/or release said chondrocytes over time and/or upon implantation.

The bone ingrowth undersurface may be used for long term fixation of the tabs or rim. That is, whereas it is important for the surgery to secure the implant to the joint surface in the most desirable corrective location, it is also important in some embodiments to prepare the anatomic undersurface of bone by abraiding it, removing about 0.5 mm of cortical bone so as to expose the underlying oxygen, blood, and nutrients of the patient to the undersurface of the implant that can gradually become incorporated into the limb bone. As this healing occurs over the course of weeks and months to one year post operation, the localized tacking sites may become less relevant and potentially inert. Thus, in some embodiments, the implant may comprise a biodegradable (bioresorbable) polymer or other material. The couplers may additionally and/or alternatively be biodegradable. Once the implant is in place, it will serve to at least one of: pad defects, cushion the joint, and restore the original damage to the joint components. The end goal is to apply minimally morbid treatment that will refurbish arthritic limb regions, leaving only the small skin scar and remote memory of the healed physical mishap.

Undersurface implant materials may involve used of the art and science from Artelon or Gore-Tex research, as each has advantages and limitations. Several implant options per joint damage area may be available to enjoy the primary surgeons manipulation to fit the clinically recovery requirements best.

In some embodiments the implant comprises a ingrowth patch on at least one of the first portion configured to engage the femur head, the second portion configured to engage the acetabulum, the side portion, and the appendage. The ingrowth patch may be configured to encourage and/or promote tissue ingrowth, such as bone ingrowth, for non-limiting example. The patch may be as large as the portion itself (whether the first portion the second portion, the side portion, or the appendage) or may be smaller than the portion (such as in the shape of a strip or other shaped patch). The ingrowth patch may comprise a surface irregularity or roughness. The ingrowth patch may be Velcro-like. In some embodiments the implant comprises an ingrowth patch on the first portion and/or the second portion, from (and in some embodiments including) a first appendage to a second appendage. In some embodiments, wherein the appendages loosen from attachment from the bone (by design and/or from wear and/or over time), the ingrowth patch aids in securing the implant to the bone. In some embodiments, the ingrowth patch comprises beads and/or bead-like elements attached to the implant. Such an ingrowth patch may be configured to simulate trabecular bone space of a normally cancellous latticework. In some embodiments, the beads are sintered beads of various sizes. In some embodiments, the beads are sintered beads about 400 microns in size. With respect to bead size, the term “about” can mean ranges of 1%, 5%, 10%, 25%, or 50%. In some embodiments, the femur head and/or the acetabulum is roughened to acquire a bleeding bone to facilitate ingrowth. In some embodiments, about 0.5 mm of cortical tissue is removed to facilitate ingrowth.

In some embodiments, the appendage of the implant comprises a hook. In some embodiments the hook is angled. The hook may comprise a piece of metal sandwiched between two polymer pieces. The hook may comprise a piece of metal encased in polymer. In some embodiments, the hook may comprise a piece of metal and a portion of the metal piece may be encased in polymer. In some embodiments, the hook may comprise a piece of metal and a portion of the metal piece may be sandwiched between two polymer pieces. The metal of the hook may reinforce the appendage tabs for securing the implant to the bone of the joint. In some embodiments, the metal of the hook is formed of a 1 centimeter by 1 centimeter metal piece. The metal of the hook, or a portion thereof, may protrude from the appendage. The metal may be bent toward the bone to which it is configured to attach. The metal may be bent at about a 270 degree angle (as compared to the non-bent portion of the metal, or as compared to the rest of the appendage, for non-limiting example). The term about when referring to angle of bend of the metal of the hook can mean variations of 1%, 5%, 10%, 20%, and/or 25%, or variations of 1 degree, 5 degrees, 10 degrees, 15 degrees, 20 degrees, 25 degrees, 30 degrees, 40 degrees, 45 degrees, and/or up to 90 degrees. In some embodiments, the bone may be prepared to receive the hook, such as by a hole or slot into which the hook (or a portion thereof) is placed. In some embodiments, the bone is not prepared in advance to receive the hook, and the hook may self-seat into the bone by pressure applied to the hook into the bone. In some embodiments, the implant may comprise multiple appendages, and a plurality of the appendages have hooks.

In some embodiments, the implant comprises a second appendage coupling the balloon to the femur head of the joint. In some embodiments, the implant comprises a second appendage coupling the balloon to at least one acetabulum of the joint. In some embodiments, the implant comprises a second appendage configured to couple at least one of the first portion, the second portion, and the side portion to at least one of the femur head and at least one acetabulum of the joint. In some embodiments, the first appendage and the second appendage are configured to provide ligamentary-like support to the femur head and the at least one acetabulum of the joint. In some embodiments, the first appendage and the second appendage are configured to provide ligamentary-like support to the joint. In some embodiments, the first appendage and the second appendage are configured to provide tendon-like support to the femur head and the at least one acetabulum of the joint. In some embodiments, the first appendage and the second appendage are configured to provide tendon-like support to the joint.

In some embodiments, the implant comprises an inflation port in communication with the interior of the balloon for inflation of the interior of the balloon with the first inflation medium. In some embodiments, the balloon is punctured to inflate the interior of the balloon with the first inflation medium. In some embodiments, the balloon is self-sealing. In some embodiments, the balloon is self-sealing upon inflation of the interior of the balloon with the first inflation medium. In some embodiments, the implant comprises a seal capable of closing the interior of the balloon.

The implant interior is filled with filler material (or an inflation medium) which aids in maintaining the desired implant dynamics within the joint structure. The nature of the filler material such as a fluid and the characteristics of the walls may be selected to maintain a desired spacing between the walls in order to accommodate the pressure applied by the bones of the joint structure to the implant and to allow suitable motion between the first and second walls of the implant which facilitate bone motion which mimics or approximates normal movement for the joint members involved. Alternatively, as mentioned above, the inner chamber may be filled with resilient material to provide the desired spacing, pressure accommodation, while allowing desired physiologic motion between implant layers. The implant is preferably configured to be shaped like the joint space and bone surfaces being replaced or to fill the void produced by injury or disease so that the natural joint spacing and cushioning of the joint interface is restored toward normal physiologic appearance and function. Fluids such as saline, mineral oil and the like may be employed to inflate the implant.

The implant interior (balloon interior) may be inflated with gas. The implant interior (balloon interior) may be inflated with liquid. The implant interior (balloon interior) may be inflated with saline. The implant interior (balloon interior) may be inflated with suspended stem cells. The implant interior (balloon interior) may be inflated with gel. The implant interior (balloon interior) may be inflated with a viscolubricant. The inflation medium in some embodiments stays within the balloon, or a portion thereof (as where there are multiple chambers to the balloon). In some embodiments, balloon contents disburse through microporosities and/or dissolving membranes into the joint. In some embodiments, balloon contents disburse by expulsive or evacuation precipitated through an implant wall after pressure from limb use. In some embodiments, balloon contents disburse by expulsive or evacuation precipitated through an implant wall from planned osmosis. In some embodiments, balloon contents disburse by expulsive or evacuation precipitated through an implant wall from vacuole rupture (whether mechanical rupture, ultrasound, or chemical rupture, for non-limiting example). In some embodiments, balloon contents disburse by expulsive or evacuation precipitated through an implant wall thereby distributing contents of the implant interior to joints as lubricious, analgesic, anti-inflammatory and/or otherwise healing substances.

In some embodiments, the implant comprises an inflation medium that is compressible. In some embodiments, the implant comprises an inflation medium that comprises a viscolubricant. In some embodiments, the implant comprises an inflation medium that comprises a pharmacologic substance. In some embodiments, the implant comprises an inflation medium that comprises an NSAID. In some embodiments, the implant comprises an inflation medium that comprises chondrocytes. In some embodiments the implant is configured to anneal the outer most layer of the implant (or a portion thereof) to the peripheral of succinct cartilage defects so as to cover them, allowing for healing. In some embodiments the implant is configured to anneal the outer most layer of the implant (or a portion thereof) to the peripheral of succinct cartilage defects so as to cover them, allowing for healing once new chondrocytes have been installed.

In some embodiments the implant may comprise vacuoles of pharmacologic substances. The vacuoles may be on a bone-engaging portion of the implant. In some embodiments, the implant comprises bubbles comprising an active substance such as a pharmacologic substance or other active substance. In some embodiments, the implant comprises spaces filled with an active substance such as a pharmacologic substance or other active substance. The implant may deliver by dissolution of the implant material (i.e. a biodegradable polymer which releases the active substance), and/or by release through pores of the implant (wherein the polymer is permeable to the active substance), and/or by fracture of the vacuole (or bubble, or space) by a catalyst such as ultrasound or pressure or other fracturing catalyst. The implant may deliver the active substance at a time after the actual implanting of the implant into the joint, for example an hour later, less than a day later, a day later, less than a week later, a week later, less than a month later, and/or a month later. In some embodiments, stem cells that are percolating in the bubble (or vacuole, or space) may be delivered to the joint space (or a constituent of the joint) after the implant is inserted into the joint. Active agents may, for non-limiting example, include stem cells, growth factors, antibiotics, and/or viscolubricants. In some embodiments, the implant may comprise enzyme absorptive ‘microscopic sponges’ that could be sucked out or evacuated at or around the time of implant delivery to the joint.

Linear or curvilinear movement between the first and second walls may result from movement of the femur head relative to the acetabulum. Rotational movement about the bone axis between the first and second walls may result from axial rotation between the femur head and acetabulum. There may be slippage between the acetabulum and the second wall in addition to wall movements within the implant per se to provide desired joint movements. The skirt is designed to secure the general implant to the joint structure so as to avoid dislocation of the implant. Movement of the joint with the implant in place will be a shared function of both the moving opposing walls of the implant but also a function of the movement of the wall which may be less attached to the joint members. There may be slight movement between the skirt, wall and the femur head. In some embodiments, one side of the side wall is in compression while the other is stretched to accommodate bone interface movement. The walls may be thicker is some areas to accommodate particular loads and the side wall may be thinner and more elastic to accommodate rolling and stretching thereof.

The interior of implant is adjustably filled by the physician from an appropriate source thereof after the implant is deployed to ensure that the pathologic joint space becomes a resilient cushion again which aids restoration of worn or damaged cartilage interfaces in the joint by covering cartilage defects with the implant material, cushioning the joint and defects therein and delivering cell regeneration agents. In one embodiment, the arthroplasty implant comprises a bio-compatible inflatable member that is filled with a biocompatible fill material such as a gas, liquid, gel or slurry, or fluid that becomes a resilient solid to provide relative movement between the first and second walls. The filling or inflation media may be inserted through an injection valve site leading to the cannula which delivers the material into the interior of the implant. In an alternative embodiment, the implant may be filled with or have an interior formed of biologically compatible resilient material, e.g. a closed cell sponge filled with suitable fluid that is inserted into the interior of the implant prior to the implant's deployment or injected into the interior after the implant is deployed at the joint site. The interior of the implant may be provided with lubricious material to facilitate movement between the inner wall surfaces and to minimize contact wear therebetween. The polymeric walls of the implant may be impregnated with or otherwise carry tissue regeneration agents such as stem cells, living chondrocytes, and/or genes to repair joint surfaces.

The walls of the implant may be (in whole and/or in part) bioabsorbable. The balloon may be (in whole and/or in part) bioabsorbable. As used herein the terms bioabsorbable, bioerodable, and/or bioabsorbable may be used interchangeably. The walls of the implant may release a pharmaceutical agent or an biological agent (such as stem cells, living chondrocytes, gene therapies, and the like). The release of such agents (whether biological or pharmaceutical, or a combination thereof) may occur over time, as the wall of the implant (or as the balloon) bioabsorbs in some embodiments, or as the joint is used (i.e. through pressure, for non-limiting example). In some embodiments, at least one of the implant walls is permeable to a pharmaceutical agent and/or a biological agent, such as in an embodiment wherein the inflation medium comprises the pharmaceutical agent and/or biological agent. In some embodiments, at least one of the implant walls has pores through which the pharmaceutical agent and/or the biological agent may fit, such as in an embodiment wherein the inflation medium comprises the pharmaceutical agent and/or biological agent.

In some embodiments, the implant comprises amniotic membrane (and/or a component thereof). In some embodiments, the implant comprises amniotic sac (and/or a component thereof). In some embodiments, the implant comprises amniotic tissue (and/or a component thereof). Amniotic membrane (and/or sac and/or tissue) is unique in that its mechanical properties include that it slippery on one side (lubricious, low modulus of elasticity) and sticky (adherent) on the other.

In some embodiments, at least one of the first wall, the second wall and the side wall comprise amniotic membrane or a component thereof In some embodiments, at least one of the first wall, the second wall and the side wall comprise amniotic sac or a component thereof In some embodiments, at least one of the first wall, the second wall and the side wall comprise amniotic tissue or a component thereof The amniotic membrane and/or amniotic sac and/or amniotic tissue may be used in conjunction with other biologic agents, pharmaceutical agents, and/or therapeutic agents. Amniotic tissue is used extensively in pleuripotential cells. It qualifies as HTBP (Human Tissue Based Product) because of the short term time span on the product and origin.

In some embodiments, the interior comprises a plurality of inflatable chambers. In some embodiments, the interior comprises a plurality of individually inflatable chambers. In some embodiments, a first chamber of the plurality of individually inflatable chambers is adapted to be inflated with the first inflation medium, and a second chamber of the plurality of individually inflatable chambers is adapted to be inflated with a second inflation medium.

In some embodiments, the first inflation medium imparts rigidity in the implant. In some embodiments, the first inflation medium imparts cushion in the implant. In some embodiments, the inflation medium chosen for the first inflation medium, and/or the particular choice of chamber (in embodiments having multiple chambers) filled with such first inflation medium aligns the joint. In some embodiments, the inflation medium chosen for the first inflation medium, and/or the particular choice of chamber (in embodiments having multiple chambers) filled with such first inflation medium aligns the bones of the joint. In some embodiments, the inflation medium chosen for the first inflation medium, and/or the particular choice of chamber (in embodiments having multiple chambers) filled with such first inflation medium changes the bone alignment. In some embodiments, the inflation medium chosen for the first inflation medium, and/or the particular choice of chamber (in embodiments having multiple chambers) filled with such first inflation medium improves joint alignment. In some embodiments, the inflation medium chosen for the first inflation medium, and/or the particular choice of chamber (in embodiments having multiple chambers) filled with such first inflation medium restores, at least in part, joint alignment. In some embodiments, individual chambers of the interior may be selectively inflated with a first inflation medium and/or a second inflation medium. In some embodiments, individual chambers of the interior are selectively inflated with a first inflation medium and/or a second inflation medium in order to reconstruct the joint and/or bones of the joint.

In some embodiments the inflation medium comprises living chondrocytes.

In some embodiments, the interior comprises a honeycomb structure. In some embodiments, the interior comprises a mesh structure. In some embodiments, the interior comprises a sponge structure.

In some embodiments a chamber of the implant is configured to receive a solid piece configured to restore joint and/or bone alignment. In some embodiments a chamber of the implant is configured to receive a rigid piece configured to restore joint and/or bone alignment. In some embodiments a chamber of the implant is configured to receive a semi-rigid piece configured to restore joint and/or bone alignment. In some embodiments, the chamber is configured to receive a plurality of solid pieces, each of which can be used to increase the space between a femur head and the acetabulum in order to restore and/or improve joint and/or bone alignment. The solid pieces may be wedge-shaped, or be provided in various sizes and/or shapes. The solid pieces may individually or together be used in a chamber or multiple chambers of the implant. The solid piece (or pieces) may be used to ratchet adjacent bones to a desired distraction and/or alignment to restore and/or improve joint and/or bone alignment. The solid piece may be put in a chamber of the implant, which may enclose or partially enclose the piece to hold the piece in place. In some embodiments, a block of biocompatible material (such as PMMA or another bone-like substitute) may be provided and may be formed (by carving or other forming method) by the surgeon to a desired shape. The formed piece may then be put in a chamber of the implant, which may enclose or partially enclose the piece to hold the piece in place. The solid (or rigid or semi-rigid pieces, depending on the need) piece may be used to fill in damage to a joint structure.

The implant interior (balloon interior) may be inflated with methymethacrylate as a liquid that becomes a solid or semi-solid (rigid or semi-rigid). In some embodiments, the inflation medium is a methyl methacrylate or other biocompatible hardening substance which can flow when initially put into the chamber, and hardens to become a rigid piece (or solid piece). The methyl methacrylate or other biocompatible hardening substance may conform to the shape of the chamber, or may conform to the shape of a space between bones and/or other joint structures. The methyl methacrylate or other biocompatible hardening substance may conform to a form chosen by the surgeon using tools and/or pressure to influence the final shape of the rigid piece formed by the methyl methacrylate or other biocompatible hardening substance upon hardening.

The solid piece (or rigid piece or semi-rigid piece—whether formed in situ or by a surgeon or pre-formed) may be cushioned by the implant. The implant may comprise an inflatable chamber between the solid piece and the femur head. The implant may comprise an inflatable chamber between the solid piece and the acetabulum. The implant may comprise a pad between the solid piece and the femur head as a cushion. The implant may comprise a pad between the solid piece and the acetabulum as a cushion.

The solid piece may provide at least one of about 1 degree of joint correction, about 2 degrees of joint correction, about 3 degrees of joint correction, about 4 degrees of joint correction, about 5 degrees of joint correction, about 6 degrees of joint correction, about 7 degrees of joint correction, about 8 degrees of joint correction, about 9 degrees of joint correction, and about 10 degrees of joint correction. With respect to degrees of joint correction, the term “about” can mean ranges of 1%, 5%, 10%, 25%, or 50%.

The implant can be used in a variety of joints where the implant replaces a bone on bone surface and cushions the interaction between the articular ends of any two bones, such as at the femoral-acetabular interspace of a patient's hip. Where the implant is substituting or enhancing articular cartilage, the rigidity can be reduced or enhanced to maximize conformation changes that arise during motion as enabled by the two opposing walls and intended inner space, coupled with considerations in any joint surgical reconstruction with accommodation to or amplification of the existing joint ligaments, tendons or dearth thereof The implant may be deflated and removed by minimally invasive surgery, for example, after the implant has served its purpose of regenerating tissue or if another clinical condition warrants its removal. However, it may not be clinically necessary to remove the implant even if inflation is lost, since the two remaining functions of patching the injured cartilage, and delivering restorative cells may justify implant retention.

The implant is inserted by minimally invasive surgery, in some embodiments, however, in other embodiments, the implant may not be inserted by minimally invasive surgery. In some embodiments, the implant is delivered through an incision that is about 0.5 inches long. In some embodiments, the implant is delivered through an incision that is about 1 centimeter long. In some embodiments, the implant is delivered through an incision that is at most about 1 inch long. In some embodiments, the implant is delivered non-arthroscopically through an incision that is at least 1 centimeter long. In some embodiments, the implant is delivered through an incision that is at most about 0.75 inches long. In some embodiments, the implant is delivered through an incision that is at most about 0.5 inches long. In some embodiments, the implant is delivered through an incision that is about 8 centimeters long. In some embodiments, the implant is delivered through an incision that is about 9 centimeters long. In some embodiments, the implant is delivered through an incision that is about 10 centimeters long. In some embodiments, the implant is delivered through an incision that is about 11 centimeters long. In some embodiments, the implant is delivered through an incision that is about 12 centimeters long. In some embodiments, the implant is delivered through an incision that is over about 10 centimeters long. In some embodiments, the implant is delivered through an incision that is at up to about 40 centimeters long. In some embodiments, the implant is delivered through multiple incisions. With respect to incision length, the term “about” can mean ranges of 1%, 5%, 10%, 25%, or 50%.

In some embodiments the implant is configured to be delivered to the joint arthroscopically. In some embodiments, the implant is configured to fit within a cannula having a distal end inner diameter of at most 10 millimeters. In some embodiments, the implant is configured to fit within a cannula having a distal end inner diameter of at most 9 millimeters. In some embodiments, the implant is configured to fit within a cannula having a distal end inner diameter of at most 5 millimeters.

In some embodiments, the implant is configured to fold in order to fit within a cannula having a distal end inner diameter of at most 10 millimeters. In some embodiments, the implant is configured to fold in order to fit within a cannula having a distal end inner diameter of at most 9 millimeters. In some embodiments, the implant is configured to fold in order to fit within a cannula having a distal end inner diameter of at most 5 millimeters.

In some embodiments, the implant is configured to be delivered to a joint through a cannula having a distal end inner diameter of at most 10 millimeters. In some embodiments, the implant is configured to be delivered to a joint through a cannula having a distal end inner diameter of at most 9 millimeters. In some embodiments, the implant is configured to be delivered to a joint through a cannula having a distal end inner diameter of at most 5 millimeters.

In some embodiments the implant is configured to be delivered to the joint arthroscopically. In some embodiments, the implant is configured to fit within a cannula having a distal end inner diameter of at most about 10 millimeters. In some embodiments, the implant is configured to fit within a cannula having a distal end inner diameter of at most about 9 millimeters. In some embodiments, the implant is configured to fit within a cannula having a distal end inner diameter of at most about 5 millimeters. With respect to cannula distal end inner diameter, the term “about” can mean ranges of 1%, 5%, 10%, 25%, or 50%.

In some embodiments, the implant is configured to fold in order to fit within a cannula having a distal end inner diameter of at most about 10 millimeters. In some embodiments, the implant is configured to fold in order to fit within a cannula having a distal end inner diameter of at most about 9 millimeters. In some embodiments, the implant is configured to fold in order to fit within a cannula having a distal end inner diameter of at most about 5 millimeters. With respect to cannula distal end inner diameter, the term “about” can mean ranges of 1%, 5%, 10%, 25%, or 50%.

In some embodiments, the implant is configured to be delivered to a joint through a cannula having a distal end inner diameter of at most about 10 millimeters. In some embodiments, the implant is configured to be delivered to a joint through a cannula having a distal end inner diameter of at most about 9 millimeters. In some embodiments, the implant is configured to be delivered to a joint through a cannula having a distal end inner diameter of at most about 5 millimeters. With respect to cannula distal end inner diameter, the term “about” can mean ranges of 1%, 5%, 10%, 25%, or 50%.

In some embodiments the implant may be provided as a deflated balloon for insertion into the joint space. In some embodiments the implant may be provided as folded balloon that may be collapsed like an umbrella for insertion into the joint space. In some embodiments the implant may be provided as collapsed balloon that is of an irregular folded pattern to minimize its folded (or collapsed) size for insertion into the joint space. In some embodiments, the implant is configured to blow up (or expand) to take the form of the expanded, distracted, debrided joint.

In some embodiments, the implant replaces periosteum.

In some embodiments, the implant is implanted to preserve bone as compared to a typical arthroplasty procedure of the joint. In some embodiments, the implant is implanted to preserve cartilage as compared to a typical arthroplasty procedure of the joint. In some embodiments, the implant is implanted with minimal soft tissue dissection as compared to a typical arthroplasty procedure of the joint. In some embodiments, the implant is implanted without joint dislocation. In some embodiments, once implanted, the joint is adaptable to revision surgery. In some embodiments once implanted, the joint retains at least one of: about 90% of normal joint function, about 95% of normal joint function, about 85% of normal joint function, about 80% of normal joint function, about 75% of normal joint function, about 70% of normal joint function, about 65% of normal joint function, about 60% of normal joint function, about 55% of normal joint function, about 50% of normal joint function, at least 95% of normal joint function, at least 90% of normal joint function, at least 85% of normal joint function, at least 80% of normal joint function, at least 75% of normal joint function, at least 70% of normal joint function, at least 65% of normal joint function, at least 60% of normal joint function, at least 55% of normal joint function, at least 50% of normal joint function, about 50%-about 75% of normal joint function, about 50%-about 70% of normal joint function, about 60-about 70% of normal joint function, about 70%-about 80% of normal joint function, about 70%-about 90% of normal joint function, about 80%-about 95% of normal joint function, about 80%-about 90% of normal joint function, and about 90%-about 95% of normal joint function. As used herein with respect to percentage of normal joint function, the term “about” can be ranges of 1%, 5%, 10%, or 25%. For example, a range of 1% with respect to about 90% of normal joint function covers 89% to 90% of normal joint function.

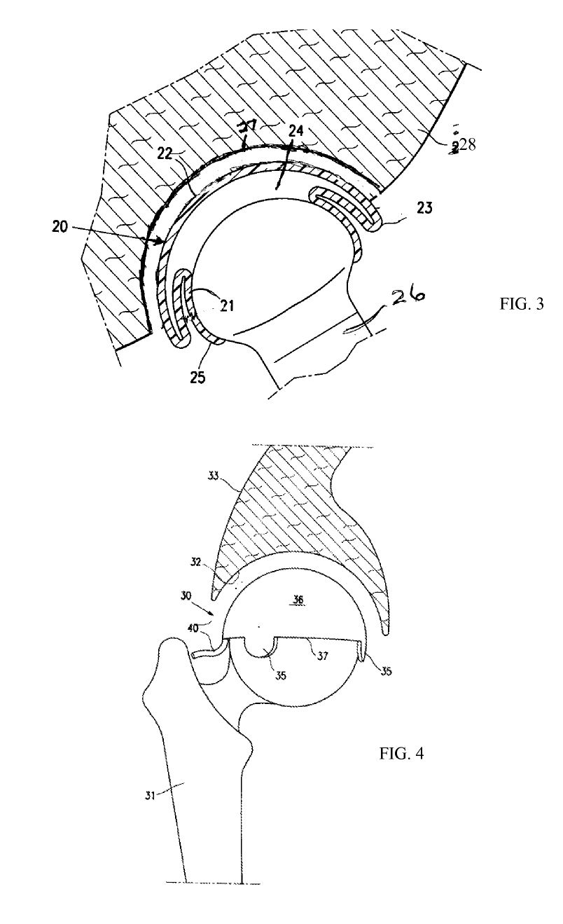

FIG. 1 is a perspective view, partially in section, illustrating a hip implant 20. The upper portion of the implant 20 has a first wall 21, a second wall 22 and a side wall 23 which define at least in part the interior 24. Skirt 25 depends from the first wall 21 and secures the first wall 21 to the end of the patient's femur 26 as best shown in FIGS. 2 and 3. FIG. 2 is an elevational view of the implant shown in FIG. 1 mounted on the head of a patient's femur. FIG. 3 illustrates the implant mounted on the head of the femur 26 with the second wall 22 of the filled upper portion configured to engage the corresponding acetabulum 27 of the patient's pelvic bone 28. FIG. 3 is a cross-sectional view of the implant shown in FIGS. 1 and 2 deployed between the head of a patient's femur and acetabulum after release of traction to allow for the bones to settle into their natural albeit pathologic angles of repose. The skirt 25 surrounds the head of the patient's femur 26 and secures the implant 20 thereto. In this embodiment, the enlarged upper portion of the implant creates overlapping layers, like a redundant membrane, in the side wall 23 between the first and second walls 21 and 22 to accommodate the normal movement of the first or second. This provides greater motion between the femur and the acetabulum and also provides implant stabilization over the head of the femur 26. This structure also accommodates variation in individual joints that occur from patient to patient.

In the embodiment shown in FIGS. 1-3 the first wall 21 does not extend across the entire end of the patient's femur. However, the implant 20 may be designed so that first wall 21 may extend over the head of the femur (and FIGS. 4-9 discussed hereinafter). The second wall 22 and the side wall 23 tend to roll as the femur 26 moves within the acetabulum 27.

In some embodiments, prior to deploying the implant embodying features of the invention, the cartilage lining the joint is prepared by removing hyaline or fibro cartilage flaps or tears, and areas of chondral advanced fissuring are excised or debrided to create precisely defined defects surrounded by stable normal remnant hyaline cartilage with vertical edges in relation to the damaged surface. It is these defects of the cartilage previously normal surface into which new living cells may be injected or otherwise inserted, and allowed to aggregate by the implant interpositional arthroplasty proximate expanded compressive external wall material. Synovitis invading the joint periphery may be vaporized and extracted conventionally or by the use of steam. Areas of greater cartilage damage are removed for subsequent regeneration and the less afflicted areas having stable cracks are treated to seal or weld the cracks. Areas where the tugor or consistency or minimally damaged cartilage can be preserved are intentionally saved rather than destroyed so as to support the normal spacing and gliding opportunity of the more normal joint interface. Thus, normal cartilage is left behind and abnormal cartilage is removed with the implant making up for the deficiencies. With the present invention, it is preferred in some embodiments to avoid joint dislocation so as to preserve natural innervations and vascularity and thus preserving the blood supply afforded by the medial and lateral circumflex arteries for the hip joint to the femoral head.

Joint preparation is usually performed under a brief general anesthetic of outpatient surgery. A muscle relaxant combined with traction (e.g. 60 pounds force for a hip implant) opens the joint wider to permit improved visualization for joint preparation and implant installation, increasing the space between the remnant cartilage from about 3 up to about 12 mm Increasing the space allows the surgeon to wash out noxious enzymes, to remove invasive synovitis, to remove loose bodies, to prepare osteochondral defects ideally and otherwise prepare the joint for the implant. Partial or complete inflation of the implant will usually precede release of traction. In some embodiments, regeneration agents or cells are inserted with the implant or as a fluid or 3-D template prior to release of traction and wound closure. It is preferred, in some embodiments, to perform joint debridement, implant deployment and application of cell regeneration agent, e.g. stem cell application, under the same anesthetic. As described by several companies in the Stem Cell Summit held in New York, N.Y. on Feb. 17, 2009, it is desirable to obtain an aspiration of the patient's bone marrow from the iliac crest after anesthesia is induced at the beginning of the operation. The intraoperative technologist will “dial in the cells” to regenerate areas of maximum pathophysiology while the surgeon debrides or otherwise prepares the joint and inserts the implant, placing the cells at the best time. Cell implantation may also occur as a secondary or tertiary reconstructive treatment adjunct.

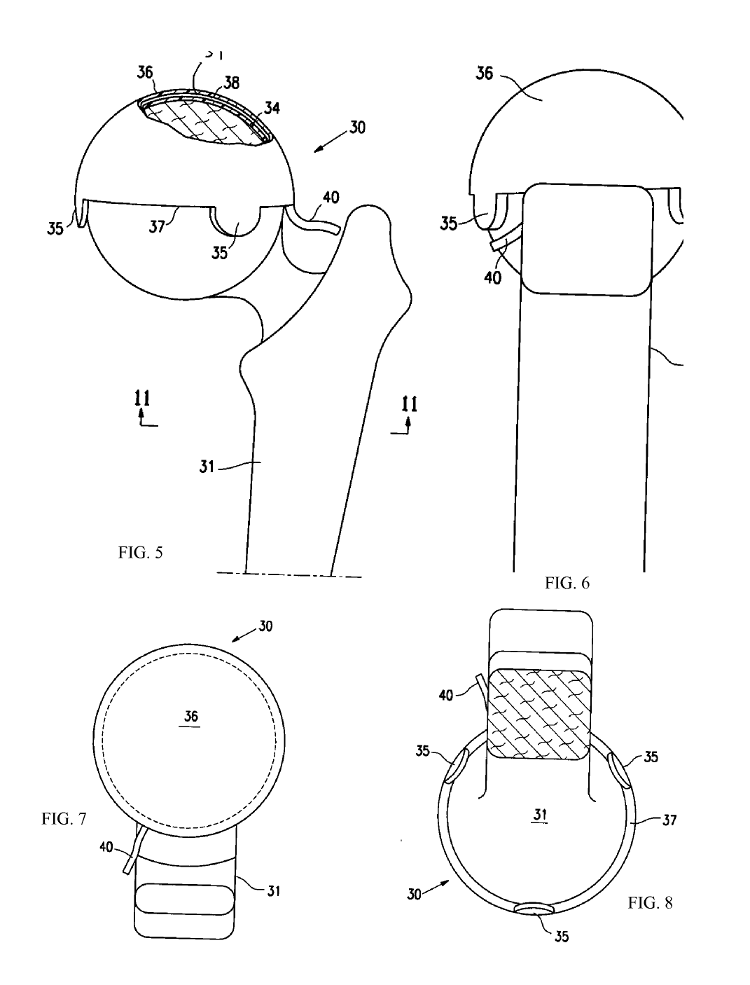

FIG. 4 is an elevational view, partially in section, of an alternative resilient implant 30 deployed within a patient's hip structure comprising the head of the patient's femur 31 and the acetabulum 32 of the patient's pelvic hip bone 33. The upper portion of the implant 30 is smaller than that shown in FIGS. 1-3. Details of the interior of the joint are not provided such as cartilage, ligaments and the like for the purpose of clarity. The resilient implant 30 embodying features of the invention is disposed within the space between the femur 31 and the acetabulum 32. FIGS. 4-8 illustrates the implant 30 mounted on the head of femur 31 without the pressure from the acetabulum 32 for purposes of clarity. FIG. 5 is an elevational anterior view of a left proximal femur with an implant placed over the femoral head portion of the hip joint as shown in FIG. 4, in partial cross section, to illustrate details thereof FIG. 6 is a lateral elevational view of a femur with the implant shown in FIG. 4, as viewed from the “side of the body” or lateral hip aspect. FIG. 7 is a superior view of a femur with the implant shown in FIG. 4. FIG. 8 is an inferior view of the hip joint invention iteration or implant in FIG. 7.

The implant 30 shown in FIGS. 4-9 is shaped like a half an orange rind or a hemisphere for a hip joint. The implant 30 has a first wall 34 seen in FIG. 5 which is secured to the head of the femur 31 by a plurality of depending tabs 35 (or appendages). The tabs 35 may be attached to the femur 31 by a suitable adhesive or mechanically such as by a screw or pin. The second wall 36 of the implant engages the acetabulum 32, but it also may be provided with tabs and the like for securing the second wall the acetabulum 32.

In some embodiments, the implant comprises a valve. The valve may be part of a wall of the implant, or part of the balloon or a portion thereof, or it may join the tube or conduit to the balloon, or it may be located subcutaneously for periodic use via injection and/or aspiration.

The side wall 37 extends between the first and second walls 34 and 36 to form an interior 38 which receives filling material 39 through tube 40 (also called a conduit herein, or may be called an inflation port). In some embodiments, the inflation port is not a tube, but is a valve which may or may not extend from a wall of the implant. The valve may be part of a wall of the implant, or part of the balloon or a portion thereof. In some embodiments, the inner diameter of the inflation port (or tube) is 5 millimeters maximum. In some embodiments, the inner diameter of the inflation port is about 1 millimeter. In some embodiments, the inner diameter of the inflation port is about 2 millimeters. In some embodiments, a needle (of typical needle sizes) may be used to inflate the implant.

In many embodiments the implant 30 (or a portion thereof, such as the balloon or balloon) is a weight bearing spacer that will allow joint motions to approach normal, whether filling the space left by an entirely collapsed peripheral joint bone or the space of ablated cartilage proximate surfaces diffusely as in osteoarthritis or succinctly as in osteonecrotic defects or localized trauma. The walls 34 and 36 may be used as a membrane for holding living cells in proximity of the osteochondral defect long enough for the cells to attach (e.g. 24 hours) or to deeply adhere (up to 28 days) or return to normal (up to one year). Weight bearing will be expected to increase as distal lower extremity joints are treated.

Motion is believed to be primarily between the spaced walls (or portions) of the implant peripherally secured to joint structures, although some motion may occur between the implant and the joint surfaces (as with current bipolar hip hemiarthroplasties). As shown in FIG. 9, the implant 30 may be provided with a slot 41 extending from the periphery 42 of the implant to a centrally located passage 43 through the implant to accommodate the ligament of the head of the femur for hip implants. FIG. 9 is a superior or cephalad view of a patient's hip with a resilient implant having features of the invention, viewed from the head of the patient or from a cephalad to caudad direction. Implant walls 34 and 36 should have sufficient inherent flexibility to mold to the existing deformities imposed by either natural ligament, bone, tendon and remaining cartilage deformities of the internal joint space filled as a cushion. The wall exteriors may be flat or formed with random or specific patterns for purposes of glide or trends for traction against adjacent surfaces, or as sulci or venues for cell delivery materials.

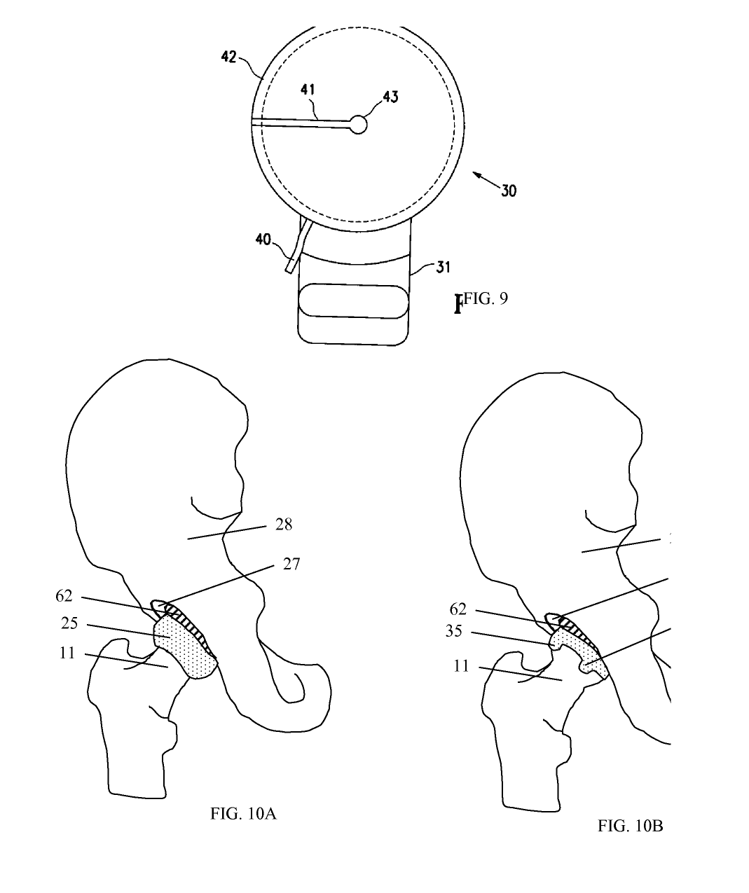

FIG. 10A depicts an embodiment of the implant having an appendage that is in the form of a skirt 25 and a balloon 62 that is mounted on a femur head 11 and implanted in the space between the femur head 11 and the acetabulum 27 of the pelvic bone 28. FIG. 10B depicts an embodiment of the implant having appendages 35 (of a tab type) and a balloon 62 that is mounted on a femur head 11 and implanted in the space between the femur head 11 and the acetabulum 27 of the pelvic bone 28.

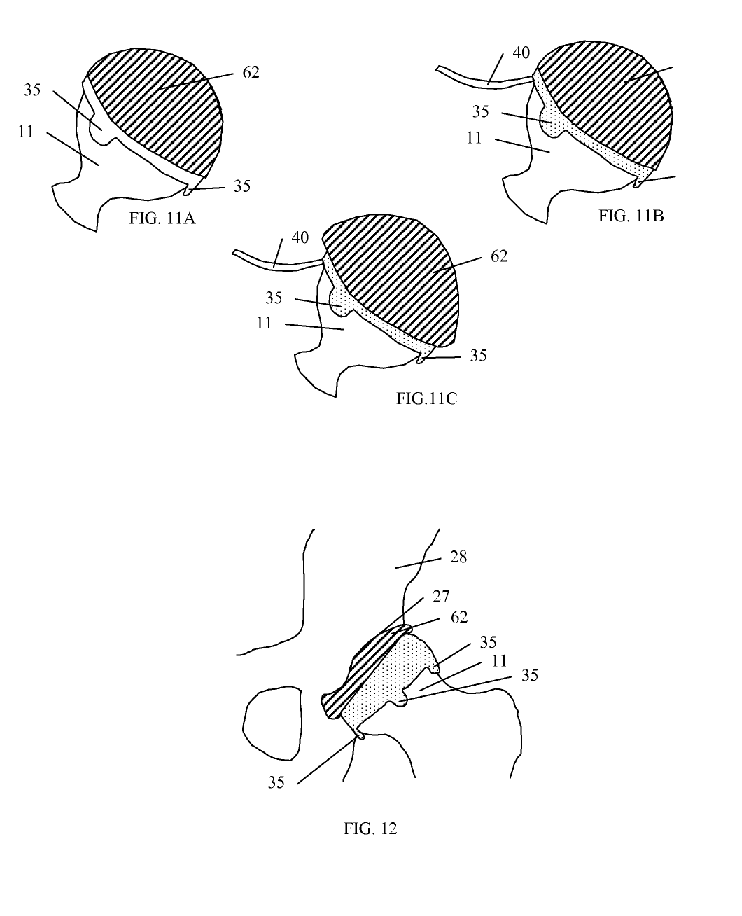

FIG. 11A depicts an embodiment of the implant having appendages 35 (tab type) and a balloon 62 that is mounted on a femur head 11 wherein the balloon 62 is minimally inflated (or not inflated).

A separate portal or tube (not shown) or the existing conduit 40 (tube or valve), may be used to extract noxious inflammatory enzymes that can be aspirated at appropriate clinical intervals. Inflammatory enzymes in the COX1, COX2 and or 5LOX pathways can be extracted. FIG. 11B depicts an embodiment of the implant having appendages 35 (tab type) and a balloon 62 that is mounted on a femur head 11 wherein the balloon 62 is minimally inflated (or not inflated) and showing an tube 40 that may be used to inflate the balloon 62 of the implant in some embodiments, or to extract inflammatory enzymes, for example.

In some embodiments, an inflation medium that generates heat (by means of a catalyst reaction or other means) may be used to delver heat to a joint structure. The heat may aide hyaline cartilage annealing. Thermal effects of the implant materials are calculated accordingly to benefit and protect the joint surface analogous to a dry suit or wet suit for a scuba diver exposed to temperature extremes. Embodiments of the implant generally seek to avoid head from friction via lubricious coatings whether allograph as amniotic membrane or polymer, for non-limiting example.

Viscolubricants can be injected into the interior of the resilient arthroplasty device through existing conduit 40 or through a long needle to aide in distension, expansion, lubrication (with predetermined microporosity). FIG. 11C depicts an embodiment of the implant having appendages 35 (tab type) and a balloon 62 that is mounted on a femur head 11 wherein the balloon 62 is inflated and showing an inflation tube 40.

In some embodiments, the first inflation medium imparts rigidity in the implant. In some embodiments, the first inflation medium imparts cushion in the implant. In some embodiments, the inflation medium chosen for the first inflation medium, and/or the particular choice of chamber (in embodiments having multiple chambers) filled with such first inflation medium aligns the joint. In some embodiments, the inflation medium chosen for the first inflation medium, and/or the particular choice of chamber (in embodiments having multiple chambers) filled with such first inflation medium aligns the bones of the joint. In some embodiments, the inflation medium chosen for the first inflation medium, and/or the particular choice of chamber (in embodiments having multiple chambers) filled with such first inflation medium changes the bone alignment. In some embodiments, the inflation medium chosen for the first inflation medium, and/or the particular choice of chamber (in embodiments having multiple chambers) filled with such first inflation medium improves joint alignment. In some embodiments, the inflation medium chosen for the first inflation medium, and/or the particular choice of chamber (in embodiments having multiple chambers) filled with such first inflation medium restores, at least in part, joint alignment. In some embodiments, individual chambers of the interior are selectively inflated with a first inflation medium and/or a second inflation medium. In some embodiments, individual chambers of the interior are selectively inflated with a first inflation medium and/or a second inflation medium in order to reconstruct the joint and/or bones of the joint.

In some embodiments, the interior comprises a honeycomb structure. In some embodiments, the interior comprises a mesh structure. In some embodiments, the interior comprises a sponge structure.

The dimensions of the various implant walls will vary depending upon the material properties thereof as well as the needs for a particular joint. Additionally, the first and second walls may require a thickness different from the side wall. Generally, the implant may have a wall thicknesses of about 0.125 mm to about 3 mm, preferably about 0.5 mm to about 1.5 mm The spacing between the first and second wall within the interior can vary from about 0.5 mm to about 5 mm for most joints.

The method of insertion for the hip joint invention will be a minimally invasive approach, ideally arthroscopically facilitated, as long as the surgical timing and result quality permit smaller incisions. The hip patient will be placed in the lateral decubitus position (lying non-operative side down on the operating table) with a stabilizing operating table pole and pad apparatus positioned to fix the pelvis. The external stabilizing table and attachments will include a padded metal pole beneath the pubis or pelvic bone from posterior to anterior, along with other external anterior and posterior pelvic stabilizing paddles. The affected leg will be attached beneath the knee with a distracting mechanism that applies about 60 pounds of distal force to open the hip joint about 1 cm once the patient is under general anesthesia. The hip joint is arthroscopically debrided through at least one anterior 0.5 cm incision and one posterior 0.5 cm incision, to remove from the femoral head acetabular (ball and socket) joint arthritic debris such as synovitis, loose bodies and noxious inflammatory enzymes. In certain cases a larger open incision may be needed. A smoothing or electronic/ultrasonic/steam or other chondroplasty method may be performed to make the remaining cartilage smoother to better accommodate the hip implant, and protuberant osteophytes or lateral bone overgrowths may be arthroscopically removed or if needed by open excision. A lateral hip incision may be required between 2 and 10 centimeters in length to deal with deformities and/or to insert the implant. In cases of major deformities appropriate reconstruction will add to the basic procedure.

Once the joint is open and cleared, the hip implant will be inserted laterally and fixed via the skirt or tabs or at least one appendage to the adjacent structures including the peripheral femoral head and/or acetabular rim. Preferably, the implant is inserted arthroscopically through a cannula about 10 mm in diameter with the implant in the deflated construct, and once inside the prepared joint space and secured therein by the skirt or tabs, the implant will be distended or inflated with gas, gel, fluid or fluid that becomes a resilient solid to fill the original natural space of about 0.5 cm between the upper acetabulum and lower femoral head, covering as much of the upper hip joint as required as the implant expands to fit the space. Tensioning will be by the surgeon's sense of proper pressure application aided by a gauged syringe for insertion of viscolubricants such as Synvisc, Hyalgan, Supartz and/or analgesics such as lidocaine gel. The insertion of liquids to the joint per se may be directly, through a cannula to the joint space previously in place for debridement, and or via a cannula or tube that is not part of the original implant assembly. Once the joint is cleaned, the implant is inserted and appropriately fixed to avoid extrusion or dislocation thereof This may be via attachment of the implant tabs and/or by a combination of tab use plus intended friction created by implant surface coverings (analogous to Velcro) or a draw string at the smaller base of the implant.

In some embodiments the attachment tabs are positioned on the implant to both secure the implant to the joint components, and to enable a physician to ensure the implant has a minimum amount of slack that could create wrinkles or loose areas to avoid unnecessary friction and/or wear of the implant or the patient's anatomy. Figures depicted herein show examples of attachment tabs configured for these dual purposes. In some embodiments, fewer tabs are needed to achieve these goals. FIG. 12 depicts an embodiment of the implant having appendages 35 (tab type) and an inflated balloon 62 that is mounted on a femur head 11 and implanted in the space between the femur head 11 and the acetabulum 27 of the pelvic bone 28. In some embodiments, more tabs are needed to achieve these goals. In some embodiments, where slack or voids exist, the balloon under compression may fill such areas. The implant in some embodiments is configured to allow hyaline and/or cartilage cells to fill any irregularities or craters in the joint components and grow to refurbish natural joint contour.

Inflation may also be specified by clinical need, and modifications in the implant multi-cell (multi-compartment) construction allows for selective inflation with substances ranging from gas to solid, including gels or semi-solids that can as part of material layered integrity either provide calculated hardness (durometer) to overcome and resist limb adjacent bone mal-alignment, and/or to deliver new regenerative tissues for restoration of natural anatomy of time. That is, certain sections of the implant may be electively inflated of left without expansion, to adjust to fit as matching a normal or uninjured contralateral limb for the involved patient.

In some situations, the removal of the implant may be needed, and embodiments of the implants described herein are configured for removal artrhoscopically, and with the allowance to perform all regular older routine accepted techniques ranging from joint debridement to drilling, partial or total replacement. In some embodiments the implant is configured for removal and replacement with a replacement implant—either immediately (within a week), or after a period of longer time (for example, after about 6 weeks to 1 year in the case of infection once all foreign bodies are removed and depending upon the surgeon's and/or infectious disease consultant's opinion).

In some embodiments, the implant comprises polymer. Polymers may comprise at least one of: a polyurethane (such as, for example, ChronoFlex AR), a polycarbonate urethane, a thermoplastic polycarbonate urethane (such as Bionate 55), ethylene-vinyl acetate copolymer, multiblock copolymers of poly(ethylene oxide) (PEO) and poly(butylene terephthalate) (PBT), PEG, PEO, and a polyetheylene. The implant may comprise to a plurality of layers of polymer (such as ChronoFlex AR) in a solvent and evaporating the solvent after applying each layer. Implants may comprise polymers such as (but not limited to) Bionate, ChronoFlex, or ChronoPrene. In some embodiments, the implant comprises a polyurethane that is sprayed and dried (wherein the spraying and drying is repeated at least once) to a desired thickness.

Adjunct therapies may be used such as viscolubricants and cells.

In some embodiments, the implant is created by dip molding a mandrel having a shape of a bone of the hip joint (the femur head and/or the acetabulum) into a polymer solution (for non-limiting example, a urethane polymer such as Chronoflex). Following each dip, the implant is dried for a specified time, which may be, for example, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 45 seconds, about 1 minute, about 2 minutes, about 5 minutes, about 10 minutes, about 15 minutes, and over about 15 minutes. The term “about” used herein in reference to drying time of the implant can mean variations of at least one of 5%, 10%, 25%, and 50%, In some embodiments, no drying step is used. The dipping may be repeated multiple times. In some embodiments a single dip is sufficient. In some embodiments, the dipping is repeated 2 times. In some embodiments, the dipping is repeated 3 times. In some embodiments, the dipping is repeated 4 times. In some embodiments, the dipping is repeated 5 times. In some embodiments, the dipping is repeated 6 times. In some embodiments, the dipping is repeated 7 times. In some embodiments, the dipping is repeated 8 times. In some embodiments, the dipping is repeated 9 times. In some embodiments, the dipping is repeated 10 times. In some embodiments, the dipping is repeated 11 times. In some embodiments, the dipping is repeated 12 times. In some embodiments, the dipping is repeated 13 times. In some embodiments, the dipping is repeated 14 times. In some embodiments, the dipping is repeated 15 times. In some embodiments, the dipping is repeated 16 times. In some embodiments, the dipping is repeated 17 times. In some embodiments, the dipping is repeated 18 times. In some embodiments, the dipping is repeated 19 times. In some embodiments, the dipping is repeated 20 times. In some embodiments, the dipping is repeated 21 times. In some embodiments, the dipping is repeated 22 times. In some embodiments, the dipping is repeated 23 times. In some embodiments, the dipping is repeated 24 times. In some embodiments, the dipping is repeated 25 times. In some embodiments, the dipping is repeated over 25 times. In some embodiments, the dipping is repeated a sufficient number of times to create an implant that is a prescribed thickness. The thickness may vary depending on the polymer and depending on the embodiment of the implant. The thickness may be at least one of: about 25 microns thick, about 50 microns thick, about 100 microns thick, about 125 microns thick, about 150 microns thick, about 200 microns thick, about 250 microns thick, about 300 microns thick, about 350 microns thick, about 400 microns thick, about 25-50 microns thick, about 50-100 microns thick, about 50-200 microns thick, about 100-150 microns thick, about 150-300 microns thick, about 100-300 microns thick, about 100-500 microns thick, about 200-500 microns thick, and about 200-1000 microns thick. The term “about” used herein in reference to thickness of the implant can mean variations of at least one of 5%, 10%, 25%, and 50%, The thickness may vary at different locations of the implant. In some embodiments, the implant is fabricated in two pieces, one or more of which is molded to form an interior when the two pieces are put together. In some embodiments, the implant is filled by puncturing the implant wall and sealing the puncture hole with a plug, patch or other sealant. The plug, patch, or other sealant may comprise Chronoflex material, for non-limiting example. The plug, patch, or other sealant may comprise the same material from which the implant is constructed, for non-limiting example.

The walls of the implant embodying features of the invention may be composite structures. For example, the innermost layer may be impervious to preclude escape of inflation or other filling media, a central layer may be porous or otherwise contain treatment or cell regeneration agents, and the outer layer may be a thin, but strong layer of a thermoplastic, such as a thermoplastic polyurethane for non-limiting example, which has microporosity sufficient to allow passage or egress of treatment or cell regeneration agents from the central layer (or second layer). The degree of microporosity to enable egress of treatment or cell regeneration agents from the central layer is found in polymer layers such as Chronoflex or Bionate 55. The external wall (and/or the bone engaging surface) of the implant may be coated and/or impregnated with a latticework of polymer that is surface sprayed or layered on the outside (or bone engaging surface) of the implant to promote cartilage tissue regeneration. This most external surface coating may contain living chondrocytes (for example, as is provided in the Carticel procedure by the Genzyme company), and/or may contain stem cells with directed gene mutations to enhance adherence of the coating to the implant. The bone engaging surface may comprise peaks and troughs. The living cells may be imposed in between (and/or provided in the) troughs of the implant surface while the surface areas of prominence (the peaks of the surface) may be used for at least one of: space validation, traction, and cell protection.

The implant embodying features of the invention may be used in a series of treatments wherein the first treatment involves use of autologous or minimally manipulated allograph interpositional tissues or xenograph, the second treatment involves the use of the same type of tissue added to stem cells or chondrocytes and the third treatment involving deployment of the implant if the first two fail or are ineffective.

The implant may be provided with latticework or other reinforcing strands, preferably on the exterior or within the wall thereof to control the maximum expansion of the implant when deployed at the orthopedic site.

The degree of distraction required in the hip joint will depend both on the nature anatomy and located pathophysiology that must be accommodated on a case by case basis and said distraction may be a combination of body position using gravitational forces and/or superimposed distracting devices.

Provided herein is a method for restoring a hip joint comprising: providing an implant configured for deployment between a femur head and acetabulum of a joint, the implant comprising a balloon comprising a first portion that is configured to engage the femur head of the joint, a second portion that is configured to engage the acetabulum of the joint, a side portion connecting the first portion and the second portion, in which the side portion facilitates relative motion between the first portion and the second portion, and an interior that is optionally inflatable with a first inflation medium; and coupling a first appendage of the balloon to the femur head of the joint.

In some embodiments, at least two of first portion, the second portion, and the side portion are contiguous. In some embodiments, the first portion comprises a first wall, the second portion comprises a second wall, and the side portion comprises a side wall.

In some embodiments the method comprises providing an ingrowth patch on at least one of the first portion configured to engage the femur head, the second portion configured to engage the acetabulum, the side portion, and the appendage. The ingrowth patch may be configured to encourage and/or promote tissue ingrowth, such as bone ingrowth, for non-limiting example. The patch may be as large as the portion itself (whether the first portion the second portion, the side portion, or the appendage) or may be smaller than the portion (such as in the shape of a strip or other shaped patch). The ingrowth patch may comprise a surface irregularity or roughness. The ingrowth patch may be Velcro-like. In some embodiments the implant comprises an ingrowth patch on the first portion and/or the second portion, from (and in some embodiments including) a first appendage to a second appendage. In some embodiments, wherein the appendages loosen from attachment from the bone (by design and/or from wear and/or over time), the ingrowth patch aids in securing the implant to the bone. In some embodiments, the ingrowth patch comprises beads and/or bead-like elements attached to the implant. Such an ingrowth patch may be configured to simulate trabecular bone space of a normally cancellous latticework. In some embodiments, the beads are sintered beads of various sizes. In some embodiments, the beads are sintered beads about 400 microns in size. With respect to bead size, the term “about” can mean ranges of 1%, 5%, 10%, 25%, or 50%. In some embodiments, the femur head and/or the acetabulum is roughened to acquire a bleeding bone to facilitate ingrowth. In some embodiments, about 0.5 mm of cortical tissue is removed to facilitate ingrowth.

In some embodiments, the method comprises coupling a second appendage of the balloon to the femur head of the joint. In some embodiments, the method comprises coupling a second appendage of the balloon to the acetabulum of the joint. In some embodiments, the method comprises coupling a second appendage of at least one of the first portion, the second portion, and the side portion to at least one of the femur head and the acetabulum of the joint. In some embodiments, coupling at least one of the first appendage and the second appendage provides ligamentary-like support to the femur head and the acetabulum of the joint. In some embodiments, coupling at least one of the first appendage and the second appendage provides ligamentary-like support to the joint. In some embodiments, the first appendage and the second appendage are configured to provide tendon-like support to the femur head and the acetabulum of the joint. In some embodiments, the first appendage and the second appendage are configured to provide tendon-like support to the joint.

In some embodiments, the method comprises providing an inflation port in communication with the interior of the balloon for inflation of the interior of the balloon with the first inflation medium. In some embodiments, the method comprises using an inflation port of the implant that is in communication with the interior of the balloon to inflate the interior of the balloon with the first inflation medium. In some embodiments, the method comprises puncturing the balloon to inflate the interior of the balloon with the first inflation medium. In some embodiments, the method comprises providing a balloon having self-sealing capability. In some embodiments, the method comprises providing a balloon having self-sealing capability upon inflation of the interior of the balloon with the first inflation medium. In some embodiments, the method comprises providing a balloon comprising a seal capable of closing the interior of the balloon.

In some embodiments, the method comprises providing a balloon having an interior comprising a plurality of inflatable chambers. In some embodiments, the interior comprises a plurality of individually inflatable chambers. In some embodiments, the method comprises inflating a first chamber of the plurality of inflatable chambers with a first inflation medium. In some embodiments, the first chamber and the inflation medium is selected based on the particular needs of the patient. For non-limiting example, if the patient has bone loss due to an injury, the chamber may be selected at the location of the missing bone, and may be filled with a rigid inflation medium (or one that becomes rigid once in the chamber) in order to replace the missing and/or damaged bone. Alternatively, or in addition, a chamber may be chosen to restore alignment of the joint, and inflated with an appropriate inflation medium to impart both alignment and cushion to the joint. In some embodiments, the method comprises inflating a second chamber of the plurality of individually inflatable chambers with a second inflation medium.

In some embodiments, the balloon is a composite structure. In some embodiments, the balloon comprises layers of porous and/or non-porous materials, or otherwise contain treatment or cell regeneration agents. In some embodiments, a first layer of the balloon is a thin, but strong layer of a thermoplastic, such as a thermoplastic polyurethane, for non-limiting example, which has microporosity sufficient to allow passage or egress of treatment or cell regeneration agents from a second layer. The second layer may be a central layer (which lies between the first layer and a third layer or a fourth layer or more layers). The first layer may comprise a bone engaging surface in some embodiments. The degree of microporosity to enable egress of treatment or cell regeneration agents from the second layer is found in polymer layers such as Chronoflex or Bionate 55. The bone engaging surface of the implant may be coated and/or impregnated with a latticework of polymer that is surface sprayed or layered on the bone engaging surface of the implant to promote cartilage tissue regeneration. This bone engaging surface coating may contain living chondrocytes (for example, as is provided in the Carticel procedure by the Genzyme company), and/or may contain stem cells with directed gene mutations to enhance adherence of the coating to the implant. The bone engaging surface may comprise peaks and troughs. The living cells may be provided in troughs while the surface peaks may be used for at least one of: space validation, traction, and cell protection.

In some embodiments, the first inflation medium imparts rigidity in the implant. In some embodiments, the first inflation medium imparts cushion in the implant. In some embodiments, the inflation medium chosen for the first inflation medium, and/or the particular choice of chamber (in embodiments having multiple chambers) filled with such first inflation medium aligns the joint. In some embodiments, the inflation medium chosen for the first inflation medium, and/or the particular choice of chamber (in embodiments having multiple chambers) filled with such first inflation medium aligns the bones of the joint. In some embodiments, the inflation medium chosen for the first inflation medium, and/or the particular choice of chamber (in embodiments having multiple chambers) filled with such first inflation medium changes the bone alignment. In some embodiments, the inflation medium chosen for the first inflation medium, and/or the particular choice of chamber (in embodiments having multiple chambers) filled with such first inflation medium improves joint alignment. In some embodiments, the inflation medium chosen for the first inflation medium, and/or the particular choice of chamber (in embodiments having multiple chambers) filled with such first inflation medium restores, at least in part, joint alignment. In some embodiments, individual chambers of the interior are selectively inflated with a first inflation medium and/or a second inflation medium. In some embodiments, individual chambers of the interior are selectively inflated with a first inflation medium and/or a second inflation medium in order to reconstruct the joint and/or in order to reconstruct bones of the joint.

Over time, ingrowth of repair tissue aids in fixation and stability externally to the implant, while the soft cushioning implant interior will absorb forces across the joint surfaces and permit proper motion. The tugor or wall tension of the implant as well as the inside distension of the implant per se can be adjusted by adding or removing the inflation substance to the implant's interior space.

Accordingly, the present invention provides a new approach to arthroplasty that involves a resilient implant device deployed between bones of the joint. In certain embodiments, the implants will need to accommodate all the normal body functional pressures and complex space movements. When in the hip joint, the normal flexion up to 120 degrees, extension of 20 degrees, abduction of 50 degrees, internal and external rotation of 45 degrees will produce variable axial, shear, and cyclic loads which the implant by design will accommodate and endure as up to 6 times body weight, consistent with a tire on a car that allows for cyclic loads different when driving straight or turning corners. The implant embodying features of the present invention provides more physiologic motion and shock absorption within the joint and has combined characteristics of anatomic design symmetry, balanced rigidity with sufficient attachment connections to adjacent normal structures, and durability that meet the needs of joint reconstruction.

The opposing internal surfaces of the first and second walls of the invention may either move together in synchrony or in opposite directions from one another (e.g. the superior wall moving medially in the hip and the inferior wall moving laterally). Optionally, the implant may be fixed to a concave surface of the joint (e.g., the acetabular hip cup) or to a convex surface of the joint (e.g. the dorsal femoral head surface), to both, or to neither (e.g., having an interference fit within the joint with an expanding balloon or cushion that fills the existing space). The implant may be inserted arthroscopically like a deflated balloon and then inflated through a cannula into the ankle or hip (or other joint structure) to act as a cushion or renewed interface for painless and stable limb motion. When feasible joint capsular and adjacent ligament tissue as well as bone will be left in place to preserve the natural body, unless interfering with reconstructed limb function.

The application of steam in addition to removing damaged debris, can smooth out and reform the joint surface. The high temperature of the steam tends to weld cracks or fissures which can be present in the cartilage surface of a damaged joint. Smoothing of joint surface cartilage with steam welds or seals existing cracks or flaps in the cartilage, especially superficially as the lamina splendors, which melt together to provide a white shiny gliding joint surface. In cases where bone is exposed, the steam can be used to stabilize the periphery of the defect in the joint surface via capsulorrhaphy or joint tightening. Open mechanical and chemical debridement may also be employed to prepare the surfaces for the implant. Some methods may include preoperative mapping by scans or arthroscopy may aide intended deployment of tissues, polymers, and other contents.

Once the implant is secured to the femoral head by means of the skirt or tabs, an impregnated transfer medium or cell template may be used, as described by Histogenics and Tygenix chondrocytes delivery systems wherein the position of concentrated cells is mechanically placed about the implant at areas of greatest cartilage damage to promote regrowth, or as in Carticel wherein watery cells are implanted beneath a periosteal membrane (a wall of the implant serving as the membrane), prior to completion of the inflation or expansion of the implant. At syringe or gauged device with measured screw-home pressure is used to inflate the implant.

Once the joint is ready to receive the implant, the deflated implant is advanced through the diaphragm of a delivery cannula (such as the Acufex from Smith & Nephew) or through the open incision site into the joint. It can be inflated by the attached cannula using a common syringe, inserting several cc's of filler material. Inserted contents and locations of cell placements depend on areas of need and joint size. In the hip implant several cc's of filler material and a viscolubricant in the interior of the implant will allow distension, cushioning, and gliding movements. Cell regeneration agents are placed in the areas of greatest need.

Methods of living stem cell or chondrocyte placement depend on the lesions and specific implant construct. Direct infusion into the joint with completion of implant inflation will press the cells into the hyaline surface, whereupon they attach within the first 24 hours. As a result, the patient should remain sedentary and the joint where the implant is deployed, non-weight bearing for the first day after surgery. Deeper osteochondral defects can be treated by ‘hyper-perfusion of cells’ via either 3-D cell transfer templates, or microneedle injection as used in treatment of diabetic patients for blood sugar testing and insulin/transdermal drug delivery. In cases of osteochondritis dissecans or localized both cartilage and bone lose, bone graft may be packed into the base of the defect followed by addition cell/tissue application. The cannula attached to the implant may be sealed and detached, or left in place for periodic aspiration of noxious enzymes as for the Cox-1, Cox-2, and 5-Lox systems, followed by reinsertion of activated substances including viscolubricants, or even more cells.

Implants embodying features of the invention may be designed for permanent or temporary deployment within a joint structure. Moreover, the implant may be formed of suitable bioabsorbable materials so that the implant may be absorbed within a particular predetermined time frame. Suitable bioabsorbable materials include polylactic acid, polyglycolic acid, polycaprolactone, copolymers, blends and variants thereof. One present method of forming the implant is to apply numerous layers of polymer such as ChronoFlex AR in a solvent and evaporating the solvent after applying each layer.

The skirting or fixation tabs of the present implant prevent joint migration during use. This is in contradistinction with prior solid polymer implants that tended toward dislocation and poor post operative function.

In some embodiments, the implant is adapted to restore natural joint function. In some embodiments, the implant is adapted to preserve viable joint tissue. In some embodiments, the implant is adapted to be placed with minimal surgery as compared to joint replacement therapy currently marketed. In some embodiments, the implant is adapted to permit weight bearing post surgery within at least one of: about 1 week, within about 1 day, within about 2 days, within about 3 days, within about 4 days, within about 5 days, within about 6 days, within about 10 days, within about 2 weeks, within about 3 weeks, within about 4 weeks, within about 5 weeks, within about 6 weeks. In some embodiments, the implant is adapted to permit weight bearing post surgery after about 1 day wherein full weight bearing is allowed in about 6 weeks. As used herein with respect to weight bearing timing, the term “about” can be a range of 1 day, 2 days, or 3 days, in some embodiments. In some embodiments, the implant is adapted to be allow for faster recovery and resumption of normal activities as compared to joint replacement therapy currently marketed.

In some embodiments, the balloon (or a portion thereof) is adapted to conform to the patient's anatomy. In some embodiments, the implant (or a portion thereof) is adapted to conform to the patient's anatomy. In some embodiments, the inflation medium is adapted to absorb a force (or forces) exerted on the joint. In some embodiments, the inflation medium is adapted to absorb a force (or forces) exerted on the bones of the joint. In some embodiments, the inflation medium is adapted to absorb a force (or forces) exerted on at least one bone of the joint. In some embodiments, the balloon is adapted to absorb shocks exerted on at least one of a bone, multiple bones, a ligament of the joint, ligaments of the joint, a tendon of the joint, tendons of the joint, and the joint in general. In some embodiments, the implant is adapted to restore natural cartilage cushion with stem cells.

In some embodiments, the balloon (or a portion thereof) is adapted to renew joint space. In some embodiments, the balloon (or a portion thereof) is adapted to reducing pain as compared to the pain felt prior to the implantation of the implant. In some embodiments, the balloon (or a portion thereof) is adapted to restore joint function. In some embodiments, the implant (or a portion thereof) is adapted to renew joint space. In some embodiments, the implant (or a portion thereof) is adapted to reducing pain as compared to the pain felt prior to the implantation of the implant. In some embodiments, the implant (or a portion thereof) is adapted to restore joint function.

In some embodiments, the implant is adapted to reverse arthritis in the joint..

In some embodiments, the balloon (or a portion thereof) is adapted to be placed into a debrided limb joint arthroscopically. In some embodiments, the balloon is adapted to pad cartilage defects. In some embodiments, the balloon is inflated to cushion the joint. In some embodiments the implant is adapted to deliver stem cells to at least one of the joint and a bone of the joint. In some embodiments the implant is adapted to deliver living chondrocytes to at least one of the joint and a bone of the joint. In some embodiments, the implant is adapted to provide a new articular surface for the joint. In some embodiments, the implant is adapted to act as a spacer in the joint. In some embodiments, the implant is adapted to space the bones of the joint apart for proper joint articulation. In some embodiments, the implant is adapted to space the bones of the joint apart for reduced bone-on-bone rubbing.

While particular forms of the invention have been illustrated and described herein, it will be apparent that various modifications and improvements can be made to the invention. One alternative implant construction involves the use of an upper portion of the implant having a net-like construction and filled with balls or ball bearing like elements that are larger than the openings in the netting. The balls or ball bearing like elements provide motion to the implant. The netting and ball bearing like elements may include regeneration agents as previously discussed, and the bearing construction may be directed toward favorable implant movement balanced with content disbursement.

In some embodiments, the implant is configured to deliver a chemotherapeutic agent to a bone or other surrounding tissues.

In some embodiments, the implant is configured to be implanted in a joint previously treated with routine total joint replacement that has been removed. In some embodiments, the implant is configured to deliver a an anti-infectious medication, (for non-limiting example: antibiotics, antifungals, and/or analgesics) allowing joint motion while treating an infected joint, to be followed by either revision surgery implanting an implant as described herein, or revision total joint replacement when infection is cleared. In some embodiments, the methods may comprise debriding the bone of the joint, implanting an implant described herein, and in some embodiments, repeating the debriding and implanting steps with additional implants. The successive series of debridements and implanting of the implants may be warranted before definitive either cancer or infection care is accomplished.

The invention is intended primarily for human use but may be extended to mammalian use. To the extent not otherwise disclosed herein, materials and structure may be of conventional design.

Moreover, individual features of embodiments of the invention may be shown in some drawings and not in others, but those skilled in the art will recognize that individual features of one embodiment of the invention can be utilized in another embodiment. Moreover, individual features of one embodiment may be combined with any or all the features of another embodiment. Accordingly, it is not intended that the invention be limited to the specific embodiments illustrated. It is therefore intended that this invention be defined by the scope of the appended claims as broadly as the prior art will permit.

Terms such as “element”, “member”, “component”, “device”, “means”, “portion”, “section”, “steps” and words of similar import when used herein shall not be construed as invoking the provisions of 35 U.S.C §112(6) unless the following claims expressly use the terms “means for” or “step for” followed by a particular function without reference to a specific structure or a specific action. All patents and all patent applications referred to above are hereby incorporated by reference in their entirety.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A hip implant configured for deployment between a femur head and a acetabulum of a hip joint, the implant comprising
a balloon comprising
a first portion that is configured to engage the femur head of the hip joint,
a second portion that is configured to engage the acetabulum of the hip joint,
a side portion connecting the first portion and the second portion, in which the side portion facilitates relative motion between the first portion and the second portion, and
an interior that is optionally inflatable with a first inflation medium; and
a first appendage configured to couple the balloon to the femur head of the joint.

2. The hip implant of claim 1, in which at least two of first portion, the second portion, and the side portion are contiguous.

3. The hip implant of claim 1, in which the first portion comprises a first wall, the second portion comprises a second wall, and the side portion comprises a side wall.

4. The hip implant of claim 1 further comprising an inflation port in communication with the interior of the balloon for inflation of the interior of the balloon with the first inflation medium.

5. The hip implant of claim 1, in which the balloon may be punctured to inflate the interior of the balloon with the first inflation medium.

6. The hip implant of claim 5, in which the balloon is self-sealing.

7. The hip implant of claim 5, in which the balloon is self-sealing upon inflation of the interior of the balloon with the first inflation medium.

8. The hip implant of claim 5, in which the implant comprises a seal capable of closing the interior of the balloon.

9. The hip implant of claim 1, in which the interior comprises a plurality of inflatable chambers.

10. The hip implant of claim 1, in which the interior comprises a plurality of individually inflatable chambers.

11. The hip implant of claim 10, in which a first chamber of the plurality of individually inflatable chambers is adapted to be inflated with the first inflation medium, and a second chamber of the plurality of individually inflatable chambers is adapted to be inflated with a second inflation medium.

12. The hip implant of claim 11, in which the first inflation medium imparts rigidity in the implant.

13. The hip implant of claim 11, in which the first inflation medium imparts cushion in the implant.

14. The hip implant of claim 1, in which the interior comprises a honeycomb structure.

15. The hip implant of claim 1, in which the interior comprises a mesh structure.

16. The hip implant of claim 1, in which the interior comprises a sponge structure.

17. The hip implant of claim 1, comprising a second appendage coupling the balloon to the femur head of the joint.

18. The hip implant of claim 1, comprising a second appendage coupling the balloon to the acetabulum of the joint.

19. The hip implant of claim 1, comprising a second appendage configured to couple at least one of the first portion, the second portion, and the side portion to at least one of the femur head and the acetabulum of the hip joint.

20. The hip implant of one of claims 17, 18 and 19, in which the first appendage and the second appendage are configured to provide ligamentary-like support to the femur head and the acetabulum of the hip joint.

21. The hip implant of one of claims 17, 18 and 19, in which the first appendage and the second appendage are configured to provide ligamentary-like support to the hip joint.

22. The hip implant of claim 1, wherein the implant is configured to fit within a cannula having a distal end inner diameter of at most 10 millimeters.

23. The hip implant of claim 1, wherein the implant is configured to fit within a cannula having a distal end inner diameter of at most 9 millimeters.

24. The hip implant of claim 1, wherein the implant is configured to fit within a cannula having a distal end inner diameter of at most 5 millimeters.

25. The hip implant of claim 1, wherein the implant is configured to fold in order to fit within a cannula having a distal end inner diameter of at most 10 millimeters.

26. The hip implant of claim 1, wherein the implant is configured to fold in order to fit within a cannula having a distal end inner diameter of at most 9 millimeters.

27. The hip implant of claim 1, wherein the implant is configured to fold in order to fit within a cannula having a distal end inner diameter of at most 5 millimeters.

28. The hip implant of claim 1, wherein the implant is configured to be delivered to a joint through a cannula having a distal end inner diameter of at most 10 millimeters.

29. The hip implant of claim 1, wherein the implant is configured to be delivered to a joint through a cannula having a distal end inner diameter of at most 9 millimeters.

30. The hip implant of claim 1, wherein the implant is configured to be delivered to a joint through a cannula having a distal end inner diameter of at most 5 millimeters.

31. The hip implant of claim 1, in which the implant replaces periosteum.

32. The hip implant of any of claims 1-31, wherein the implant is configured to at least one of: pad cartilage, cushion the joint, deliver a pharmacologic substance, remove noxious enzymes, debride upon implantation, debride the joint following implantation, deliver a therapeutic substance, deliver a biologic substance, and deliver living stem cells.

33. The hip implant of any of claims 1-32, wherein the implant is configured to deliver a chemotherapeutic agent to a bone or other surrounding tissues.

34. The hip implant of any of claims 1-33, wherein the implant is configured to deliver an anti-infectious medication to a bone or other surrounding tissues.

35. The hip implant of any of claims 1-33, wherein the implant is configured to deliver at least one of an antibiotic, antifungals, and analgesics agent.

36. The hip implant of any of claims 1-35, wherein the implant is configured to be selectively inflated to realign limbs.

37. A method comprising: implanting a hip implant of any of claims 1-36 into a subject, wherein the implant reverses arthritis in the subject.

38. A method comprising: implanting a hip implant of any of claims 1-36 into a hip joint of a subject and treating a component of the hip joint of the subject with at least one of an allograph tissue, an autograph tissue, and an xenograph tissue.

39. The method of claim 38, wherein the implanting step is at least one of: prior to the treating step, simultaneous with the treating step, and following the treating step.

40. A method comprising: implanting a hip implant of any of claims 1-36 into a subject, wherein the implant at least one of: restores joint function and controls arthopathies.

41. The method of claim 40, wherein the implanting spares existing anatomy.

42. A method comprising: debriding a femur head of a hip joint of a subject, and implanting a hip implant of any of claims 1-36 into the hip joint of the subject, whereby the implant is configured to anneal to the cartilage of the subject.

43. The method of claim 42, wherein the debriding is achieved by steam application.

44. A method comprising: implanting a hip implant of any of claims 1-36 into a joint previously treated with a total joint replacement.

45. The method of claim 44, comprising removing the total joint replacement prior to implanting the hip implant.

46. The method of claim 44, comprising clearing infectious matter from the joint and/or surrounding tissues.

47. The method of claim 46, comprising implanting a second implant of any of claims 1-36 following removing the implant previously implanted in the joint.

48. The method of claim 46, comprising replacing the joint of the subject following removing the implant previously implanted in the joint.

49. The method of claim 44, comprising debriding the bone of the joint, and implanting an implant of any of claims 1-36.

50. The method of claim 44, comprising repeating the debriding and implanting steps.
